(19) United States
(12) Patent Application Publication
(10) Pub. No.: US 2013/0195886 A1
(43) Pub. Date: Aug. 1, 2013

(54) Methods for Fructanase and Fructokinase Inhibition

(71) Applicant: The Regents of the University of Colorado, A Body, Denver, CO (US)

(72) Inventors: Richard J. Johnson, Centennial, CO (US); Miguel A. Lanaspa-Garcia, Denver, CO (US); Stephen Dreskin, Denver, CO (US)

(73) Assignee: The Regents of the University of Colorado, A Body Corporate, Denver, CO (US)

(21) Appl. No.: 13/686,877

(22) Filed: Nov. 27, 2012

Related U.S. Application Data
(60) Provisional application No. 61/563,806, filed on Nov. 27, 2011.

(51) Int. Cl. A61K 38/14; A61K 45/00

(52) U.S. Cl. CPC A61K 38/14; A61K 45/00
USPC 424/158.1; 514/3.1

(57) ABSTRACT

Provided are methods and compositions method for inhibiting fructokinase activity within the gastrointestinal tract cell of a subject. The compositions and methods treat or prevent conditions associated with increased permeability and oxidative stress in the gastrointestinal tract of a subject.

### CROSS-REFERENCE TO RELATED APPLICATIONS

This application is related to U.S. Provisional Application 61/563,806, filed Nov. 27, 2011, which is incorporated herein in its entirety and to which priority is claimed under 35 USC 119.

### STATEMENT REGARDING FEDERALLY SPONSORED DEVELOPMENT

Development for this invention was supported in part by NIH Grant No. HL-68607. Accordingly, the United States Government may have certain rights in this invention.

### FIELD OF THE INVENTION

The present inventors have identified fructanase and fructokinase as key enzymes that ultimately drive a host of conditions characterized by increased gastrointestinal tract (gut) permeability as a function of fructose metabolism.

### BACKGROUND OF THE INVENTION

Fructokinase (also known as ketohexokinase, or KHK) is the first enzyme in fructose metabolism and catalyzes the conversion of fructose to fructose 1 phosphate. There are two major isoforms of fructokinase, consisting of fructokinase C (KHK-C) and fructokinase A.¹⁻² Due to the high Km for fructokinase A (which is approximately 28 mM), fructose is preferentially metabolized by KHK-C.¹ In turn, KHK-C is expressed primarily in the liver, intestines, and kidney.³ To date, almost all studies have focused on the role of KHK-C in the liver and kidney⁴⁻⁵, and nothing is known of the role of intestinal KHK-C in health and disease.

KHK-C is unique among sugar kinases in that its metabolism of fructose is associated with a rapid depletion of intracellular ATP.⁶ Unlike glucokinase, in which excessive phosphorylation of glucose is prevented by a negative feedback system, the metabolism of fructose by KHK-C will result in rapid phosphorylation with a fall in intracellular phosphate and ATP. As ATP levels fall, protein synthesis transiently stops and the cell develops features consistent with ischemia.⁷ The decrease in intracellular phosphate stimulates AMP deaminase which accelerates the degradation of AMP to purine products including uric acid.⁶ Intracellular uric acid levels rise, and in turn mediates intracellular oxidative stress and the production of inflammatory mediators.⁴ For example, proximal tubular cells exposed to fructose undergo ATP depletion, intracellular uric acid formation, the production of oxidants, and the synthesis of monocyte chemoattractant protein-1 (MCP-1), and this pathway can be prevented by silencing the cells for KHK.⁴ We have also found that stimulation of hepatic cells (human HepG2 cells) results in the production of oxidants that can be prevented by silencing KHK.

The administration of fructose to rats has been reported to increase intestinal permeability, with the appearance of endotoxemia that is thought to have a role in mediating the effect of fructose to induce fatty liver.⁸ These effects were not attributed to intestinal fructokinase, and to date the role of fructokinase in intestinal permeability has not yet been considered.

### BRIEF DESCRIPTION OF THE DRAWINGS

FIG. 1 shows that peanut IgE levels were higher in the group receiving fructose consistent with fructose increasing the risk for peanut IgE response (p<0.02 by Mann Whitney test).

FIG. 2 shows that mice receiving peanuts plus cholera toxin plus fructose showed a worse symptom score (P<0.05) and fall in temperature (P=0.06) compared to mice receiving peanuts with low dose cholera toxin alone. These studies document that fructose accelerates the development of IgE mediated food allergy in mice. These data show that fructose increases the risk for severe allergic reaction to peanuts.

FIG. 3 shows in vitro studies employing human intestinal epithelial cells (CaCo-2) revealed that exposure of these cells to 5 mM fructose markedly decreased the expression of genes involved in the maintenance of cell polarity. Specifically, the expression of e-cadherin, a marker of epithelial cells, is dramatically down-regulated in cells exposed to fructose for 96 hours.

FIGS. 4A-4D show that fructose causes an alteration in intestinal permeability as a consequence of fructokinase. Both WT mice and KHK-A/C KO mice drank the same amount of fructose (FIG. 4A). Quantitative real time PCR was performed for fructokinase C (KHK-C, FIG. 4B), and tight junction genes occludin (FIG. 4C) and ZO-1 (FIG. 4D) using actin as an internal control. As shown, WT mice fed fructose show an upregulation of KHK mRNA expression in association with a significant decrease in occludin and ZO-1 mRNA. These studies suggest fructose is increasing intestinal permeability.

FIG. 5 shows fructokinase C (KHK-C) is expressed throughout the intestinal tract, including the duodenum, jejunum, cecum and colon whereas it is not expressed in mice in which both fructokinase C and A have been knocked out (KHK-A/C KO).

FIG. 6 shows exemplary sequences of KHK.

### DETAILED DESCRIPTION OF THE INVENTION

The present inventors have identified that the metabolism of fructose by increased gastrointestinal tract (gut) induces inflammation in the intestinal cell walls of and increases intestinal permeability. The increased intestinal permeability, in turn, increases access of antigens to the circulation, thereby increasing the risk for IgE-mediated food allergies, IgA mediated diseases (celiac disease, IgA nephropathy) and inflammatory bowel disease, for example. The absorption and metabolism of fructose by the proximal duodenum and small bowel, for example, may also have a role in diabetes due to the production of inflammatory mediators and oxidative stress. Further, the present inventors have found that the production of fructose in the gut stemming from the metabolism of ingested fructans may also engage the intestinal fructokinase pathway to drive certain diseases in humans and animals (namely horses).

In certain aspects of the present invention, therefore, there are provided various compositions and methods for blocking fructokinase either systemically or within the intestine in the prevention and treatment of these disorders. In particular embodiments, the methods and compositions described herein comprise therapeutic agents that can specifically inhibit fructokinase C; fructokinase C, but not fructokinase A; or both fructokinase C and fructokinase A to treat or prevent various disorders.

### I. DEFINITIONS

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the terms “diabetic” or “diabetes” refers to Type 1 diabetes, wherein the pancreas produces little or no insulin; Type 2 diabetes, wherein the body becomes resistant to the effects of insulin or produces little or no insulin; or disease state occurring as sequelae of other primary diseases that include the symptoms of either or both of elevated blood sugar(hyperglycemia) and the excretion of sugar in the urine (glycosuria).

As used herein, the terms “disease,” “disorder,” or “complication” refers to any deviation from a normal state in a subject. In preferred embodiments, the methods and compositions of the present invention are useful in the diagnosis and treatment of diseases where the expression of a KHK protein differs in subjects with disease and subjects not having disease. The present invention finds use with any number of diseases including, but not limited to, renal diseases.

As used herein, by the term “effective amount” “amount effective,” or the like, it is meant an amount effective at dosages and for periods of time necessary to achieve the desired result.

As used herein, the term “preventing” means causing the clinical symptoms of the disease state not to develop, e.g., inhibiting the onset of disease, in a subject that may be exposed to or predisposed to the disease state, but does not yet experience or display symptoms of the disease state.

As used herein, the term “expression” in the context of a gene or polynucleotide involves the transcription of the gene or polynucleotide into RNA. The term can also, but not necessarily, involve the subsequent translation of the RNA into polypeptide chains and their assembly into proteins.

As used herein, the terms “interfering molecule” refer to all molecules, e.g., RNA or RNA-like molecules, that have a direct or indirect influence on gene expression, such as the silencing of a target gene sequence. Examples of other interfering RNA molecules include siRNAs, short hairpin RNAs (shRNAs), single-stranded siRNAs, microRNAs (miRNAs), and dicer-substrate 27-mer duplexes. Examples of “RNA-like” molecules include, but are not limited to, siRNA, single-stranded siRNA, microRNA, and shRNA molecules that contain one or more chemically modified nucleotides, one or more non-nucleotides, one or more deoxyribonucleotides, and/or one or more non-phosphodiester linkages. Thus, siRNAs, single-stranded siRNAs, shRNAs, miRNAs, and dicer-substrate 27-mer duplexes are subsets of “interfering molecules.” “Interfering molecules” also may include PMOs.

As used herein, the terms “phosphothioate morpholino oligomer(s),” “a PMO” or “PMOs” refer to molecules having the same nucleic acid bases naturally found in RNA or DNA (i.e. adenine, cytosine, guanine, uracil or thymine), however, they are bound to morpholine rings instead of the ribose rings used by RNA. They may also be linked through phosphorodiamidate rather than phosphodiester or phosphorothioate groups. This linkage modification eliminates ionization in the usual physiological pH range, so PMOs in organisms or cells are uncharged molecules. The entire backbone of a PMO is made from these modified subunits.

As used herein, the term “antisense sequence” refers to an oligomeric compound that is at least partially complementary to a target nucleic acid molecule to which it hybridizes. In certain embodiments, an antisense compound modulates (increases or decreases) expression of a target nucleic acid. Antisense compounds include, but are not limited to, compounds that are oligonucleotides, oligonucleosides, oligonucleotide analogs, oligonucleotide mimetics, and chimeric combinations of these.

As used herein, the term “RNA interference” (RNAi) refers to a post-transcriptional gene silencing (PGSR) process whereby one or more exogenous small interfering RNA (siRNA) molecules are used to silence expression of a target gene.

As used herein, “siRNAs” (short interfering RNAs) refer to double-stranded RNA molecules, generally around 15-30 nucleotides in length, that are complementary to the sequence of the mRNA molecule transcribed from a target gene.

As used herein, “shRNAs” (small hairpin RNAs) are short “hairpin-turned” RNA sequences that may be used to inhibit or suppress gene expression.

As used herein, a “pharmaceutical composition” or “therapeutic agent” refers to a composition comprising a KHK inhibitor and optionally a pharmaceutically acceptable diluents or carrier. In the case of an interfering molecule, for example, the interfering molecule may be combined with suitable pharmaceutically acceptable diluents, such as phosphate-buffered saline.

As used herein, the term “subject” refers to any animal (e.g., a mammal), including, but not limited to, humans, non-human primates, cattle, livestock, hoofed animals, rodents, and the like, which is to be the recipient of a particular treatment. In one embodiment, the subject is a human, and in another embodiment, the subject is a fructan-ingesting hoofed animal, and in a particular embodiment, is a horse

As used herein, the terms “treating” or “treatment” or “alleviation” refers to both therapeutic treatment and prophylactic or preventative measures, wherein the object is to prevent or slow down (lessen) the targeted pathologic condition or disorder.

### II. INCREASED INTESTINAL PERMEABILITY

In accordance with one aspect of the present invention, there is provided a method for inhibiting fructokinase activity in intestinal (gut) cells of a subject comprising administering to the subject an effective amount of a KHK inhibitor. In one embodiment, the intestinal cells comprise a member from the group consisting of duodenum, jejunum, and ileum cells of the small bowel or cecal or colonic cells of the large bowel. The effectiveness of the administered agent on intestinal permeability^{18, 35} may be measured by suitable methods in the art, including but not limited to the sucrosuria test (for duodenal permeability)^{18, 20}, the lactulose/mannitol^{18, 20}, lactulose/rhamnose¹⁹, or ⁵¹Cr-EDTA test^{33, 37-38} permeability tests (for small bowel permeability), or sucralose or polyethylene glycol (PEG) testing for colonic permeability.

In accordance with one aspect of the present invention, there is provided a method for reducing intestinal permeability and/or oxidative stress in a digestive tract of a subject associated with fructose metabolism in a subject. The method comprises administering to the subject an effective amount of a KHK inhibitor. In one embodiment, the reduced permeability and/or oxidative stress is reduced in the duodenum, jejunum, and ileum of the small bowel, or of the cecum or remainder of the large colon of the subject. In one embodiment, the administering is to treat or prevent a condition selected from the group consisting of an IgE-mediated food allergy, celiac disease, IgA nephropathy, inflammatory bowel disease, and laminitis, which will be discussed in fuller detail below.

In accordance with yet another aspect of the present invention, there is provided a method for inhibiting fructokinase activity within gastrointestinal tract cells of a subject comprising administering to the subject an effective amount of a KHK inhibitor. In one embodiment, the method further comprises administering a fructanase inhibitor in an amount effective to reduce metabolism of fructose to fructans. In another embodiment, the method may further comprise administering to the subject an amount of probiotics that consume fructose or uric acid.

In accordance with another aspect of the present invention, there is provided a method for inhibiting fructokinase activity associated with ingestion of ingestible polysaccharides containing fructans by a subject, the method comprising administering to the subject an effective amount of a KHK inhibitor. In one embodiment, the fructans are first metabolized to fructose by fructanases by the subject and then inhibited by the KHK inhibitor. In another embodiment, the method further comprises administering a fructanase inhibitor to the subject.

It is contemplated that the present invention is not limited to the disease states described herein. Aspects of the present invention are directed to compositions and methods for the inhibition of fructose metabolism in the subject. In addition, it is contemplated that other diseases, e.g., laminitis in horses, may be primarily addressed by the inhibition of fructanase.

### III. FOOD ALLERGIES

Increased intestinal permeability is a significant contributor to the development of food allergies. Small openings can occur in the lining of the intestine, which allow large molecules of undigested or incompletely digested food to enter the bloodstream. If the quantity is too great for the liver to “clear” almost immediately, the immune system has a chance to recognize these molecules as being foreign to the body and produces antibodies against them. When the food is eaten again and again passes into the bloodstream undigested or only partially digested, the antibodies bind with the food. These antibody-food complexes can travel through the bloodstream to any part of the body where they then cause problems. Furthermore, blood borne soluble allergens can bind to IgE that is bound to IgE receptors on mast cells and basophils, leading to cellular activation and allergic reactions, including anaphylaxis. There are many causes of “leaky gut” including immaturity, toxins, nutritional deficiencies, inflammatory bowel disease, poor digestion, and food allergies. There is a vicious cycle involved with these internal factors since the leaky gut also causes them or contributes to their severity.

Food allergy has increased markedly in the last decades, and affects 3-6% of children today and 2% of adults.¹⁰⁻¹¹ The four most common IgE-mediated food allergies are to cow's milk, eggs, peanut/tree nuts, and fish/shellfish.¹⁰⁻¹¹ In all food allergy syndromes, a key pathophysiological process is a breakdown in the normal mucosal barrier. Indeed, an increased intestinal permeability is an important permissive factor¹²⁻¹³, as anaphylaxis will not occur unless the food antigen is absorbed.¹⁴ Thus, one can posit that food allergy will not occur if the intestinal barrier is maintained, and as such blocking the increased intestinal permeability from sugar or fructose should both prevent and also lessen the risk for a serious anaphylactic reaction by reducing antigen absorption. In this regard, no one has ever considered the role of intestinal fructokinase in this process.¹⁵

Thus, the present inventors have developed therapeutic agents that have the ability to inhibit the metabolism of fructose in the gut will be of great benefit in the treatment of food allergies. In accordance with one aspect of the present invention, there is provided a method for inhibiting fructokinase activity in gut cells of a subject comprising administering to the subject an effective amount of a KHK-C inhibitor. In one embodiment, the gut cells comprise jejunum, duodenum, and/or ileum cells from the small bowel. In another embodiment, the KHK inhibitor may block KHK present in the large colon, including the cecum. In one embodiment, the inhibiting is done to treat or prevent an IgE-mediated food allergy.

In accordance with another embodiment of the present invention, there is provided a method for preventing, reducing or treating an allergic reaction to food, including an anaphylactic reaction, in a subject comprising administering an amount of a KHK-C inhibitor.

### III. CELIAC DISEASE

Celiac disease is an immunological disorder in which subjects develop IgA antibodies against gluten, including IgA anti-gliadin antibodies, and IgA anti-tissue glutaminase antibodies.¹⁶ The disease manifests as abdominal pain, diarrhea, and malabsorption, but some subjects are asymptomatic or have mild symptoms. The condition is associated with disease of the duodenum and jejunum, and rarely colon, and is characterized by villous atrophy and variable degrees of submucosal inflammation. Subjects who are prone to develop celiac disease often carry HLA-DQ2, documenting an important genetic risk factor.¹⁶ However, the observation that celiac disease has increased dramatically over the last several decades also suggests an environmental component. In the US, for example, there has been a doubling in prevalence of celiac disease every 15 years that cannot be simply ascribed to more sensitive diagnostic tests.¹⁷ Today celiac disease is predicted to affect 1-3 percent of the US population at some point during their lives.¹⁶

A striking aspect of celiac disease is that there is evidence for increased intestinal permeability in both the duodenum and small bowel¹⁸⁻²⁰, which are the primary sites where fructokinase is expressed.³ The duodenal permeability can be demonstrated by increase levels of sucrose in the serum and urine following a sucrose challenge compared to normal subjects.²⁰⁻²¹ Pathologicial lesions consisting of villous atrophy and submucosal inflammation also occur in the duodenum and jejunum.²²

A role for fructokinase has not every been proposed as having a role in celiac disease, but we believe that it is highly likely. Specifically, we propose that the increased prevalence of celiac disease is due to increased intestinal permeability from a fructose/sugar fructokinase reaction in the intestinal wall that leads to increased absorption of gluten antigens in the blood in response to gluten-enriched foods.

Thus, therapeutic agents that have the ability to inhibit the metabolism of fructose in the gut will be of great benefit in the treatment of celiac disease. In accordance with one aspect of the present invention, there is provided a method for preventing or treating celiac disease in a subject comprising administering to the subject an effective amount of KHK inhibitor. In one embodiment, the administering of a KHK inhibitor reduces the amount of IgG antibodies against gluten, IgG anti-gliadin antibodies, and IgA anti-tissue glutaminase in the subject.

### IV. INFLAMMATORY BOWEL DISEASE

Crohn's disease and ulcerative colitis are classified as inflammatory bowel diseases. Crohn's disease typically affects the ileum as well as other parts of the gut, whereas ulcerative colitis is limited to the colon. Inflammatory bowel disease, and particularly Crohn's disease, have been increasing in the past decades. Crohn's disease has also been associated with increased intake of both sucrose and refined carbohydrates.²⁶⁻²⁸ Fructose intake, such as from fruits, is not.²⁶ Sugar intake has not been linked with ulcerative colitis.²⁸ While sugar intake is associated with Crohn's, there is a general belief that reducing sugar intake is not beneficial in this disease²⁹, and a role for fructokinase in the pathogenesis of inflammatory bowel disease has never been proposed.³⁰⁻³¹

Increased intestinal permeability has been shown in inflammatory bowel disease, and is characterized by upregulation of claudin 2 and downregulation of claudins 5 and 8.³² The increased intestinal permeability is thought to play a critical role in the pathogenesis of inflammatory bowel disease. In Crohn's disease the increased intestinal permeability precedes the development of the disease³³⁻³⁴ and predicts relapse.³⁵ There is evidence that blocking the intestinal permeability defect may be able to both prevent and treat the disease.³⁶ By blocking either systemic or intestinal fructokinase, intestinal permeability to sugar intake and to carbohydrates can be improved, which will both help prevent inflammatory bowel disease as well as reduce the risk for progression.

Thus, therapeutic agents that have the ability to inhibit the metabolism of fructose in the gut will be of great benefit in the treatment of Inflammatory Bowel Disease. In accordance with one aspect of the present invention, there is provided a method for preventing or treating Inflammatory Bowel Disease in a subject comprising administering to the subject an effective amount of KHK inhibitor. In one embodiment, the Inflammatory Bowel Disease is selected from group consisting of Crohn's disease and ulcerative colitis.

### V. IgA NEPHROPATHY

IgA nephropathy is the most common glomerular disease in the developed world and is associated with elevated IgA antibodies and IgA deposits in the mesangium of the kidney. This disease is also associated with increased intestinal permeability³⁷⁻³⁸ with the increased frequency of systemic IgA antibodies to food antigens.³⁹⁻⁴⁰ While there is a reduction in the local mucosal immune response, there is a hyperactive systemic IgA response, which would be compatible with increased intestinal permeability and increased exposure to dietary or other antigens introduced via the gut.⁴¹ An increased risk of IgA nephropathy has also been linked with the intake of high carbohydrate foods.⁴²⁻⁴³ By blocking the increased intestinal permeability associated with sugar intake and the western diet, blocking fructokinase will reduce the absorption of the antigens triggering the IgA response and hence this treatment should be helpful to prevent the progression of this renal disease.

Thus, therapeutic agents that have the ability to inhibit the metabolism of fructose in the gut will be of great benefit in the treatment of IgA nephropathy. In accordance with one aspect of the present invention, there is provided a method for preventing or treating IgA nephropathy in a subject comprising administering to the subject an effective amount of KHK inhibitor.

### VI. FOUNDER DISEASE

Laminitis (also known as Founder) is a disease that affects the feet of ungulates, best known in horses and cattle. Laminitis is characterized by inflammation of the digital laminae of the hoof and/or separation of the lamellae of the inner hoof and distal phalanx. It is a common cause of lameness in horses particularly, affecting 2-5% of horses.⁴⁴ Laminitis is both associated with and predicted by the presence of equine metabolic syndrome, characterized by insulin resistance, hypertriglyceridemia, hypertension, and obesity (with preferential fat deposition in the neck and tailhead).⁴⁵⁻⁴⁶ Epidemiologically, both equine metabolic syndrome and laminitis are associated with the ingestion of grasses rich in indigestible polysaccharides containing fructans.^{44, 47} Fructans are polymers of fructose and exist either as levans, which are polymers consisting of β(2→6)-linked fructosyl units with branching at the 2-1 position, or inulins, which have β(2→1) linked fructosyl units with a terminal glucose unit.⁴⁸ Fructans are present in many C3 grasses and about 15% of flowering plants, where they are stored in the stems and roots. The typical horse may ingest 15 kg of pasture grass per day⁴⁹, of which up to 40 to 50% may contain fructans, resulting in an ingestion of 5-7 kg fructan per day (amounting to 10 to 15 g/kg body wt).⁴⁷

The ingestion of fructans causes laminitis. The administration of oligofructose (10 g/kg body weight) to horses by nasogastric tube results in laminitis within 48 hours in association with acidic diarrhea, the development of both D and L-lactic acidosis, endotoxemia and glucose elevation.^{47, 50} Fecal flora is also altered, with a shift to gram positive organisms, particularly Streptococci.⁵¹ The currently held hypothesis is that laminitis is the consequence of the breakdown of fructans in the cecum by bacteria, resulting in the development of lactic acidosis and endotoxemia with the release of bacterial products that activate local metalloproteinases that break down the basement membrane between the hoof lamellae and bone.⁵² According to this theory, accelerating breakdown of fructans with enzymes might in fact be protective, as noted by a recent patent application by US 2009/0252719 to Phillipps et al. Importantly, the concept that this disease might be due to either the absorption of fructose or from metabolism of fructose by intestinal fructokinase has not been considered.

Mammals do not have the enzymes to degrade fructans. However, some gut bacteria, express fructanases that can degrade fructans to fructose. These bacteria are primarily in the Firmicute phyla, and consist primarily of gram positive bacteria such as Streptococcus salivarus, Strep mutans, Bacillus sp, Clostridia sp, and Bifidobacterium.⁵³ The horse has large numbers of bacteria in both the small and large intestine⁵⁴, with the former being the site where fructokinase is expressed. When fructans are administered to horses, there is the rapid degradation of fructans in the small bowel.⁵⁵ Thus, the administration of fructans should be expected to result in fructose generation in the gut. Consistent with this proposal, we have found that fructose levels increase in the blood of some horses eating fructan-rich pasture grasses.

The marked ingestion of fructan-rich grasses, followed by their digestion in the small bowel, might be expected to provide a bolus of fructose for absorption and metabolism. While some of the fructose would be degraded by local bacteria or absorbed into the blood stream, much of the fructose would be metabolized in intestinal cells where it would cause local inflammation and increased intestinal permeability mediated by intestinal fructokinase. While the slow absorption and metabolism of fructose from fructans would provide a mechanism for the development of equine metabolic syndrome, the large bolus effects might cause severe inflammation in the bowel with endotoxemia, and have a significant pathogenesis in laminitis. In addition, the rise in intracellular uric acid in the intestinal epithelial cell from fructokinase-dependent fructose metabolism is in part responsible for the proinflammatory and prooxidative effects.^{4, 56} By blocking either fructokinase specifically in the intestine or systemically, one should be able to both block equine metabolic syndrome and the development of laminitis.

Thus, therapeutic agents that have the ability to inhibit the metabolism of fructose in the gut will be of great benefit in the treatment of equine metabolic syndrome and laminitis. In accordance with one aspect of the present invention, there is provided a method for preventing or treating equine metabolic syndrome in a hoofed animal comprising administering to the hoofed animal an effective amount of KHK inhibitor. In accordance with one aspect of the present invention, there is provided a method for preventing or treating laminitis in a hoofed animal, e.g., cattle or livestock, comprising administering to the subject an effective amount of KHK inhibitor.

### VII. GUT BACTERIA-INDUCED OBESITY

Obese humans and laboratory animals have a typical gut flora consisting primarily of Firmicutes as opposed to Bacteroidetes.⁵⁷⁻⁵⁸ Firmicutes are the primary bacterial phyla producing fructanases⁵³, and consistent with this observation, the bacteria associated with human obesity were found to have a unique ability to metabolize indigestible polysachharides⁵⁹ (such as fructans) and to express fructose and glutamate metabolic activity.⁶⁰ Evidence that these bacteria contribute to obesity was shown by experiments in which the colonic bacteria from obese (ob/ob) mice were transferred to lean mice which resulted in the latter gaining more fat as determined by dual-energy X-ray absorptiometry.⁶⁰ Moreover, if western diet is given to mice lacking gut bacteria (germ free mice), obesity does not develop.⁶¹

The proposed mechanisms by which obesity develops from gut bacteria include increased weight due to an increase in caloric intake from the digestion of the polysaccharides^{57, 59}, or via alterations in expression of angiopoietin-like 4⁶¹ and the endocannabinoid system in the colon, the latter which results in increased gut permeability and increases endotoxin levels.⁶² Importantly, a role for fructokinase has not been previously considered. However, given that the degradation of fructans by Firmicute bacteria should generate fructose, and because fructose induced metabolic syndrome is mediated by fructose, it is believed that the inhibition of fructokinase will block the development of obesity in response to gut bacteria. Thus, in accordance with one aspect of the present invention, there is provided a method for inhibiting the development of gut bacteria-induced obesity comprising administering to the subject an effective amount of KHK inhibitor.

### VIII. BARIATRIC SURGERY

Bariatric surgery has been found to improve glycemic control/insulin resistance via an effect that cannot be attributed strictly to weight loss and which is greater with surgeries in which the duodenum and proximal small bowel are bypassed (Gastric bypass, Roux-en-Y surgery) as opposed to simple gastric banding (banded gastroplasty).⁶³ Indeed, improvement in diabetes or insulin resistance can be shown by simply bypassing the duodenum even without gastric banding.⁶⁴ Currently it is thought that the benefit of surgery on diabetes is due to the effect of the bypass procedure to allow more rapid delivery of nutrients to the ileum resulting in a rapid rise in glucagon-like peptide-1 (GLP-1).⁶⁵⁻⁶⁷ However, it is also thought that there may be some factor in the duodenum or small bowel that has not been yet identified that could provide protection from diabetes. In addition, studies in humans have shown that the bypassing of the proximal bowel leads to correction of diabetes even before weight loss occurs and is associated with an improvement in insulin resistance rather than insulin secretion; furthermore, this results in a reduction in triglycerides and an improvement in hepatic lipotoxicity.⁶⁸

Since fructokinase is heavily expressed in the duodenum and jejunum, the bypassing of this segment would result in blocking the fructokinase-metabolism of fructose in the intestinal wall. Thus, blocking fructokinase, either systemically or via the intestine, should provide similar protection against the development of diabetes or in the early treatment of diabetes without requiring the abdominal surgical procedure. Alternatively, the use of a fructokinase inhibitor might provide additional protection for subjects undergoing bariatric surgery, especially when the surgery does not involve bypassing the proximal bowel (such as gastric banding). In view of the above, there is also provided a method for treating or preventing fructose metabolism in a subject undergoing bariatric surgery comprising administering to the subject an effective amount of KHK inhibitor in accordance with an aspect of the present invention.

### IX. FRUCTANASE INHIBITION

As mentioned above, the currently held hypothesis in the case of laminitis is that the disease is a consequence of the breakdown of fructans in the cecum by bacteria, resulting in the development of lactic acidosis and endotoxemia with the release of bacterial products that activate local metalloproteinases that break down the basement membrane between the hoof lamellae and bone.⁵² Further, as mentioned, mammals do not have the enzymes to degrade fructans. However, some gut bacteria, express fructanases that can degrade fructans to fructose. These bacteria are primarily in the Firmicute phyla, and consist primarily of gram positive bacteria such as Streptococcus salivarus, Strep mutans, Bacillus sp, Clostridia sp, and Bifidobacterium.⁵³ The above discussion focused on the downstream inhibition of fructokinase so as to prevent the rapid phosphorylation of fructose during the metabolism of fructose. However, further aspects of the present invention are directed to the inhibition of fructan metabolism, such as by inhibiting the activity of fructanases in the subject.

In accordance with another aspect of the present invention, there is provided a method for reducing an amount of fructanase-producing bacteria in a subject comprising administering to the subject an amount of an antibiotic effective to reduce the amount of fructanase-producing bacteria in the subject.

In accordance with another aspect of the present, there is provided a method for preventing obesity in a subject comprising administering an amount of an antibiotic effective to reduce the amount of fructanase-producing bacteria in the subject.

In accordance with another aspect of the present invention, there is provided a method for treating or preventing laminitis in an undulate animal comprising administering an amount of an antibiotic effective to reduce the amount of fructanase-producing bacteria in the animal. In one embodiment, the animal is a horse.

In accordance with yet another aspect of the present invention, there is provided is a method for preventing or treating equine metabolic syndrome in an equine animal comprising administering to the equine animal an effective amount of an antibiotic effective to reduce an amount of fructanase-producing bacteria in the subject.

In accordance with another aspect of the present invention, there is provided a method for identifying subjects at risk for developing obesity comprising identifying an amount of fructanase-producing bacteria in the subject.

In accordance with another aspect of the present invention, the present invention is directed to a method for treating or preventing diabetes in a subject comprising administering to the subject an effective amount of a fructanase inhibitor.

Exemplary fructanase-producing bacteria targeted by the fructanase inhibitors include those from the Firmicute phyla, and consist primarily of gram positive bacteria such as Streptococcus salivarus, Strep mutans, Bacillus sp, Clostridia sp, and Bifidobacterium. Typically, fructanase-producing bacteria are gram positive bacteria. Accordingly, in one embodiment, the antibiotic for use in the present invention may be a therapeutic agent that is selective for gram positive bacteria. The classification for gram positive bacteria relies on the positive or negative results from Gram's staining method, which uses complex purple dye and iodine. Because gram-positive bacteria have more layers of peptidoglycan in their cell walls than gram-negative, they can retain the dye. Typically, the antibiotic is a antibiotic that is not absorbed into the digestive tract of the subject. In a particular embodiment, the antibiotic comprises vancomycin. In another embodiment, one could replace fructanase-secreting bacteria by administering, e.g., seeding, the gut of the subject with other non-fructanase secreting bacteria.

It is contemplated that instead of utilizing antibiotics, methods may be provided that intentionally utilize the fructan/fructose pathway to induce such physiological effects as weight gain. In one embodiment, for example, there is a method for inducing weight gain in a subject comprising administering fructans and/or fructose to induce weight gain in a subject with cachexia. The cachexia may be associated with cancer, for example.

### X. KHK INHIBITORS

The KHK inhibitor for use in the present invention may include one or more of a ribozyme, an interfering molecule, a peptide, a small molecule, or an antibody targeted to KHK (KHK-A or KHK-C). In one embodiment, the KHK inhibitor inhibits KHK-C, but does not inhibit KHK-A. By not inhibiting KHK-A, it is meant that the inhibitor is specifically targeted to inhibit the activity of KHK-C and that this results in the activity or expression of KHK-C being inhibited to a greater extent than the activity or expression of KHK-A. While not wishing to be bound by theory, it is believed that KHK-A at least metabolizes fructose less rapidly than KHK-C as indicated by its higher Km value. Further, while not wishing to be bound by theory, it is believed that due to its higher Km and its more ubiquitous distribution, KHK-A may not induce the same severity of ATP depletion or intracellular uric acid generation with fructose as seen with KHK-C. Nevertheless, in certain embodiments, the KHK-C inhibitor may inhibit KHK-C activity, as well as KHK-A activity.

KHK can be inhibited by a number of means as set forth further below, including silencing via small molecule compounds, miRNA, shRNA, sRNA, or a PMO directed to a portion of the sequence described at the genbank accession numbers provided below. See U.S. Patent Publication 20060110440 for background on sRNA silencing, the entirety of which is hereby incorporated by reference. As discussed above, therapeutic agents can be developed inhibit KHK-C, or both KHK-A and KHK-C to achieve a beneficial effect on obesity, sugar cravings and evidence of glucose-induced tubular dysfunction of the kidney.

1. Screening Methods

In accordance with one aspect of the present invention, the invention provides assays for screening test compounds which bind to or modulate the activity of a KHK polypeptide or bind to and inhibit or affect expression of a KHK polynucleotide. A test compound preferably binds to a KHK polypeptide. More preferably, a test compound decreases or increases KHK activity by at least about 10, preferably about 50, more preferably about 75, 90, or 100% relative to the absence of the test compound.

In accordance with another aspect of the present invention, there is provided a method of screening for compounds capable of differentially inhibiting KHK-C relative to KHK-A. The method comprises contacting at least one KHK inhibitor test compound with a KHK-C polypeptide. In addition, the method comprises detecting binding of said at least one KHK inhibitor test compound to said KHK-C polypeptide, wherein a test compound which binds to said KHK-C polypeptide is identified as potential KHK inhibitor agent.

In accordance with another aspect of the present invention, there is provided a method of screening for compounds capable of inhibiting KHK-C. The method comprises i) determining the activity of a KHK-C polypeptide without contact with a test compound; and ii) determining the activity of said KHK-C polypeptide upon contact with the test compound, wherein a test compound that modulates activity of said KHK-C polypeptide is identified as potential KHK inhibitor agent.

1.1. Test Compounds

Test compounds relate to agents that potentially have therapeutic activity, i.e., bind to or modulate the activity of a KHK polypeptide or bind to or affect expression of a KHK polynucleotide. Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994).

1.2. High Throughput Screening

Test compounds can be screened for the ability to bind to and inhibit KHK polypeptides or polynucleotides or to affect KHK activity or KHK gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format. Alternatively, “free format assays,” or assays that have no physical barrier between samples, can be used.

1.3. Binding Assays

For binding assays, the test compound is preferably, but not necessarily, a small molecule which binds to and occupies, for example, the active site of the KHK polypeptide, such that normal biological activity is prevented. Examples of such small molecules include, but are not limited to, small peptides or peptide-like molecules.

In binding assays, either the test compound or the KHK polypeptide can comprise a detectable label, such as a fluorescent, radioisotopic, chemiluminescent, or enzymatic label, such as horseradish peroxidase, alkaline phosphatase, or luciferase. Detection of a test compound which is bound to the KHK polypeptide can then be accomplished, for example, by direct counting of radioemission, by scintillation counting, or by determining conversion of an appropriate substrate to a detectable product.

Those skilled in the art equipped with teachings herein will appreciate that there are multiple conventional methods of detecting binding of a test compound. For example, binding of a test compound to a KHK polypeptide can be determined without labeling either of the interactants. A microphysiometer can be used to detect binding of a test compound with a KHK polypeptide. A microphysiometer (e.g., CYTOSENSOR™) is an analytical instrument that measures the rate at which a cell acidifies its environment using a light-addressable potentiometric sensor (LAPS). Changes in this acidification rate can be used as an indicator of the interaction between a test compound and a KHK polypeptide (McConnell et al., Science 257, 19061912, 1992).

In another alternative example, determining the ability of a test compound to bind to a KHK polypeptide can be accomplished using a technology such as real-time Bimolecular Interaction Analysis (BIA) (Sjolander & Urbaniczky, Anal Chem. 63, 23382345, 1991, and Szabo et al., Curr. Opin. Struct. Biol. 5, 699705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a KHK polypeptide can be used as a “bait protein” in a two-hybrid assay or three-hybrid assay (see, e.g., U.S. Pat. No. 5,283,317; Zervos et al., Cell 72, 223232, 1993; Madura et al., J. Biol. Chem. 268, 1204612054, 1993; Bartel et al., BioTechniques 14, 920924, 1993; Iwabuchi et al., Oncogene 8, 16931696, 1993; and Brent WO94/10300), to identify other proteins which bind to or interact with the KHK polypeptide and modulate its activity.

In many screening embodiments, it may be desirable to immobilize either the KHK polypeptide (or polynucleotide) or the test compound to facilitate separation of bound from unbound forms of one or both of the interactants, as well as to accommodate automation of the assay. Thus, either the KHK polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the KHK polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a KHK polypeptide (or polynucleotide) can be accomplished in any vessel suitable for containing the reactants. Examples of such vessels include microtiter plates, test tubes, and microcentrifuge tubes.

In a specific embodiment, the KHK polypeptide may be a fusion protein comprising a domain that allows the KHK polypeptide to be bound to a solid support. For example, glutathione S-transferase fusion proteins can be adsorbed onto glutathione sepharose beads (Sigma Chemical, St. Louis, Mo.) or glutathione derivatized microtiter plates, which are then combined with the test compound or the test compound and the nonadsorbed KHK polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly as described above. Alternatively, the complexes can be dissociated from the solid support before binding is determined.

Other techniques for immobilizing proteins or polynucleotides on a solid support also can be used in the screening assays of the invention. For example, either a KHK polypeptide (or polynucleotide) or a test compound can be immobilized utilizing conjugation of biotin and streptavidin. Biotinylated KHK polypeptides (or polynucleotides) or test compounds can be prepared from biotinNHS(Nhydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a KHK polypeptide, polynucleotide, or a test compound, but which do not interfere with a desired binding site, such as the active site of the KHK polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the KHK polypeptide or test compound, enzyme-linked assays which rely on detecting an activity of the KHK polypeptide, and SDS gel electrophoresis under non-reducing conditions.

Screening for test compounds which bind to a KHK polypeptide or polynucleotide also can be carried out in an intact cell. Any cell which comprises a KHK polypeptide or polynucleotide can be used in a cell-based assay system. A KHK polynucleotide can be naturally occurring in the cell or can be introduced using techniques such as those described above. Binding of the test compound to a KHK polypeptide or polynucleotide is determined as described above.

1.4. Enzyme Assays

Test compounds can be tested for the ability to increase or decrease the KHK activity of a KHK polypeptide. KHK activity can be measured such as by that described in the Examples. Enzyme assays can be carried out after contacting either a purified KHK polypeptide, a cell membrane preparation, or an intact cell with a test compound. A test compound which inhibits KHK activity of a KHK polypeptide by at least about 10, preferably about 50, more preferably about 75, 90, or 100% is identified as a potential therapeutic agent for inhibiting KHK activity. A test compound which decreases KHK activity by at least about 10, preferably about 50, more preferably about 75, 90, or 100% is identified as a potential therapeutic agent for inhibiting KHK activity.

1.5. Gene Expression

In another embodiment, test compounds, which increase or decrease KHK gene expression are identified. A KHK polynucleotide is contacted with a test compound, and the expression of an RNA or polypeptide product of the KHK polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of KHK mRNA or polypeptide expression in the cells can be determined by methods well known in the art for detecting mRNA or polypeptide. Either qualitative or quantitative methods can be used. The presence of polypeptide products of a KHK polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a KHK polypeptide.

Such screening can be carried out either in a cell-free assay system or in an intact cell. Any cell which expresses a KHK polynucleotide can be used in a cell-based assay system. The KHK polynucleotide can be naturally occurring in the cell or can be introduced using techniques such as those described above. Either a primary culture or an established cell line, such as CHO or human embryonic kidney 293 cells, can be used.

In certain embodiments, inhibiting KHK involves downregulation of gene expression, translation or activity of KHK genes. There are two isoforms of KHK relevant to therapeutic activity discussed below, as well as for screening and production of therapeutic agents: KHK-C (predominant form of KHK, Gen Bank Accession # NM₁₃ 006488 (http://www.ncbi.nlm.nih.gov/entrez/viewerfcgi?db=nucleotide&val=5670341) SEQ. ID. Nos 1 & 2 and KHK-A (Gen Bank Accession# NM_{—}000221(http://www.ncbi.nlm.nih.gov/entrez/viewerfcgi?db=nucleotide&val=455769 2) SEQ. ID. Nos. 3 & 4.

The methods and compositions described herein may be directed at inhibiting expression of inhibiting the gene expression, translation or activity of any one or more of these KHK genes. In a particular embodiment, the methods and compositions described herein are directed at inhibiting the gene expression, translation or activity of KHK.

1.6 Gene Delivery

Those skilled in the art will appreciate that numerous delivery mechanisms are available for delivering a therapeutic agent to an area of need. By way of example, the agent may be delivered using a liposome as the delivery vehicle. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes conventionally used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

2.1 Determination of a Therapeutically Effective Dose

The determination of a therapeutically effective dose of therapeutic agents identified by a screening method herein is well within the capability of those skilled in the art. A therapeutically effective dose refers to that amount of active ingredient which modulates KHK activity compared to that which occurs in the absence of the therapeutically effective dose.

Therapeutic efficacy and toxicity, e.g., ED50 (the dose therapeutically effective in 50% of the population) and LD50 (the dose lethal to 50% of the population), can be determined by standard pharmaceutical procedures in cell cultures or experimental animals. The dose ratio of toxic to therapeutic effects is the therapeutic index, and it can be expressed as the ratio, LD50/ED50.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Preferably, a therapeutic agent reduces expression of a KHK gene or the activity of a KHK polypeptide by at least about 10, preferably about 50, more preferably about 75, 90, or 100% relative to the absence of the reagent. The effectiveness of the mechanism chosen to decrease the level of expression of a KHK gene or the activity of a KHK polypeptide can be assessed such as by hybridization of nucleotide probes to KHK-specific mRNA, quantitative RT-PCR, immunologic detection of a KHK polypeptide, or measurement of KHK activity.

2.2 Conjunctive Therapeutic Agents

In any of the embodiments described above, any of the compositions of the invention can be co-administered with other appropriate therapeutic agents (conjunctive agent or conjunctive therapeutic agent) for the treatment or prevention of a target disease. Selection of the appropriate conjunctive agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects. Any of the therapeutic methods and compositions comprising a KHK inhibitor described herein can be co-administered with another conjunctive agent to a subject in need of such therapy. In one embodiment, the conjunctive agent may be one or more agents used in the prevention or treatment of an IgE-mediated food allergy, celiac disease, IgA nephropathy, obesity, inflammatory bowel disease, and laminitis, for example.

Exemplary conjunctive agents that may be formulated and/or administered with any form of a KHK-C inhibitor as described herein include, but are not limited to, angiotensin-converting enzyme (ACE) inhibitors, aldosterone antagonists, amphetamines, amphetamine-like agents, Angiotensin II receptor antagonists, anti-oxidants, aldose reductase inhibitors, biguanides, sorbitol dehydrogenase inhibitors, thiazolidinediones (glitazones), thiazide and thiazide-like diuretics, triglyceride synthesis inhibitors, uric acid lowering agents, e.g., xanthine oxidase inhibitors, antioxidants, flavonols, mitochondrial protectant agents, and combinations thereof.

Exemplary ACE inhibitors include, but are not limited to, Benazepril (Lotensin), Captopril, Enalapril (Vasotec), Fosinopril, Lisinopril (Prinivil, Zestril), Moexipril (Univasc), Perindopril (Aceon), Quinapril (Accupril), Ramipril (Altace), Trandolapril (Mavik), and combinations thereof.

Exemplary aldosterone antagonists include, but are not limited to, Spironolactone, Eplerenone, Canrenone (canrenoate potassium), Prorenone (prorenoate potassium), Mexrenone (mexrenoate potassium), and combinations thereof.

Exemplary amphetamines include, but are not limited to, amphetamine, methamphetamine, methylphenidate, p-methoxyamphetamine, methylenedioxyamphetamine, 2,5-dimethoxy-4-methylamphetamine, 2,4,5-trimethoxyamphetamine, and 3,4-methylenedioxymethamphetamine, N-ethylamphetamine, fenethylline, benzphetamine, and chlorphentermine as well as the amphetamine compounds of Adderall®; actedron; actemin; adipan; akedron; allodene; alpha-methyl-(.+−.)-benzeneethanamine; alpha-methylbenzeneethanamine; alpha-methylphenethylamine; amfetamine; amphate; anorexine; benzebar; benzedrine; benzyl methyl carbinamine; benzolone; beta-amino propylbenzene; beta-phenylisopropylamine; biphetamine; desoxynorephedrine; dietamine; DL-amphetamine; elastonon; fenopromin; finam; isoamyne; isomyn; mecodrin; monophos; mydrial; norephedrane; novydrine; obesin; obesine; obetrol; octedrine; oktedrin; phenamine; phenedrine; phenethylamine, alpha-methyl-; percomon; profamina; profetamine; propisamine; racephen; raphetamine; rhinalator, sympamine; simpatedrin; simpatina; sympatedrine; and weckamine. Exemplary amphetamine-like agents include but are not limited to methylphenidate. Exemplary compounds for the treatment of ADD include, but are not limited to, methylphenidate, dextroamphetamine/amphetamine, dextroamphetamine, and atomoxetine (non-stimulant).

Exemplary Angiotensin II receptor antagonists or angiotensin receptor blockers (ARBs) include, but are not limited to losartan, irbesartan, olmesartan, candesartan, valsartan, and combinations thereof.

Exemplary anti-oxidant compounds include but are not limited to L-ascorbic acid or L-ascorbate (vitamin C), menaquinone (vitamin K 2), plastoquinone, phylloquinone (vitamin K 1), retinol (vitamin A), tocopherols (e.g., α, β, γ and δ-tocotrienols, ubiquinol, and ubiquione (Coenzyme Q10)); and cyclic or polycyclic compounds including acetophenones, anthroquinones, benzoquiones, biflavonoids, catechol melanins, chromones, condensed tannins, coumarins, flavonoids (catechins and epicatechins), hydrolyzable tannins, hydroxycinnamic acids, hydroxybenzyl compounds, isoflavonoids, lignans, naphthoquinones, neolignans, phenolic acids, phenols (including bisphenols and other sterically hindered phenols, aminophenols and thiobisphenols), phenylacetic acids, phenylpropenes, stilbenes and xanthones. Additional cyclic or polycyclic antioxidant compounds include apigenin, auresin, aureusidin, Biochanin A, capsaicin, catechin, coniferyl alcohol, coniferyl aldehyde, cyanidin, daidzein, daphnetin, deiphinidin, emodin, epicatechin, eriodicytol, esculetin, ferulic acid, formononetin, gernistein, gingerol, 3-hydroxybenzoic acid, 4-hydroxybenzoic acid, 3-hydroxycoumarin, juglone, kaemferol, lunularic acid, luteolin, malvidin, mangiferin, 4-methylumbelliferone, mycertin, naringenin, pelargonidin, peonidin, petunidin, phloretin, p-hydroxyacetophenone, (+)-pinoresinol, procyanidin B-2, quercetin, resveratol, resorcinol, rosmaric acid, salicylic acid, scopolein, sinapic acid, sinapoyl-(S)-maleate, sinapyl aldehyde, syrginyl alcohol, telligrandin umbelliferone and vanillin. Antioxidants may also be obtained from plant extracts, e.g., from blackberries, blueberries, black carrots, chokecherries, cranberries, black currants, elderberries, red grapes and their juice, hibiscus, oregano, purple sweet potato, red wine, rosemary, strawberries, tea (e.g., black, green or white tea), and from various plant ingredients as ellagic acid.

Exemplary aldose reductase inhibitors include, but are not limited to, epalrestat, ranirestat, fidarestat, sorbinil, and combinations thereof.

Exemplary biguanides include, but are not limited to, metformin, and less rarely used phenformin and buformin, proguanil, and combinations thereof.

Exemplary thiazolidinediones include, but are not limited to, troglitazone, pioglitazone, ciglitazone, rosiglitazone, englitazone, and combinations thereof. Exemplary sorbitol dehydrogenase inhibitors are disclosed in U.S. Pat. Nos. 6,894,047, 6,570,013, 6,294,538, and US Published Patent Application No. 20050020578, the entirety of which are incorporated by reference herein.

Exemplary thiazide and thiazide-like diuretics include, but are not limited to, benzothiadiazine derivatives, chlortalidone, metolazone, and combinations thereof. Exemplary triglyceride synthesis inhibitors include, but are not limited to, diglyceride acyltransferase 1 (DGAT-1) inhibitors.

Exemplary uric acid lowering agents include, but are not limited to, xanthine oxidase inhibitors, such as allopurinol, oxypurinol, tisopurine, febuxostat, inositols (e.g., phytic acid and myo-inositol), and combinations thereof.

It is appreciated that suitable conjuvant therapeutic agents for use in the present invention may also comprise any combinations, prodrugs, pharmaceutically acceptable salts, analogs, and derivatives of the above compounds.

In one embodiment, the KHK inhibitor may be administered to the subject along with one or more other therapeutic agents that are active in acute and chronic kidney disease. Exemplary conjuvant therapeutic agents for this use include but are not limited to angiotensin-converting enzyme (ACE) inhibitors, aldosterone antagonists, Angiotensin II receptor antagonists, anti-oxidants, aldose reductase inhibitors, biguanides, sorbitol dehydrogenase inhibitors, thiazolidinediones (glitazones), and xanthine oxidase inhibitors.

In a particular embodiment, the KHK and/or fructanse inhibitors described herein may be used in conjunction with any other therapies for the treatment or prevention an IgE-mediated food allergy, celiac disease, IgA nephropathy, inflammatory bowel disease, laminitis, or any other disorder described herein. Additional therapies for the treatment of food allergies and celiac disease include but are not limited to the administration of diet regimens, vitamins, foods, or other therapeutic agents to the subject, including but not limited to multivitamin supplements, medicinal clay, papain, pyridoxal-5-phosphate, silica, vitamin B complexes, and the like. Additional therapies for the treatment or prevention of IgA nephropathy include the administration of angiotensin converting enzyme (ACE) inhibitors, angiotensin II receptor blockers (ARBs), or glucocorticoids. Additional therapies for the treatment or prevention of inflammatory bowel disease include but are not limited to anti-TNF agents, aminosalicylates, antibiotics, corticosteroids, and immune modifiers. Additional therapies for the treatment or prevention of laminitis include but are not limited to intravenous fluid therapy, systemic antimicrobials, intravenous dimethyl sulfoxide (DMSO), anti-inflammatory drugs, and the administration of mineral oil with a nasogastric tube.

It is appreciated by one skilled in the art that when any one or more the KHK inhibitors described herein are combined with an conjuvant therapeutic agent, the KHK inhibitor(s) may critically allow for increased efficacy of the conjuvant therapeutic agent or allow for reduction of the dose of the other therapeutic agent that may have a dose-related toxicity associated therewith.

The mode of administration for a conjunctive formulation in accordance with the present invention is not particularly limited, provided that the KHK inhibitor and the conjunctive agent are combined upon administration. Such an administration mode may, for example, be (1) an administration of a single formulation obtained by formulating a KHK inhibitor and the conjunctive agent simultaneously; (2) a simultaneous administration via an identical route of the two agents obtained by formulating a KHK inhibitor and a conjunctive agent separately; (3) a sequential and intermittent administration via an identical route of the two agents obtained by formulating a KHK inhibitor and a conjunctive agent separately; (4) a simultaneous administration via different routes of two formulations obtained by formulating a KHK inhibitor and a conjunctive agent separately; and/or (5) a sequential and intermittent administration via different routes of two formulations obtained by formulating a KHK inhibitor and a conjunctive agent separately (for example, a KHK or its composition followed by a conjunctive agent or its composition, or inverse order) and the like.

The dose of a conjunctive formulation may vary depending on the formulation of the KHK inhibitor and/or the conjunctive agent, the subject's age, body weight, condition, and the dosage form as well as administration mode and duration. One skilled in the art would readily appreciate that the dose may vary depending on various factors as described above, and a less amount may sometimes be sufficient and an excessive amount should sometimes be required.

The conjunctive agent may be employed in any amount within the range causing no problematic side effects. The daily dose of a conjunctive agent is not limited particularly and may vary depending on the severity of the disease, the subject's age, sex, body weight and susceptibility as well as time and interval of the administration and the characteristics, preparation, type and active ingredient of the pharmaceutical formulation. An exemplary daily oral dose per kg body weight in a subject, e.g., a mammal, is about 0.001 to 2000 mg, preferably about 0.01 to 500 mg, more preferably about 0.1 to about 100 mg as medicaments, which is given usually in 1 to 4 portions.

When a KHK inhibitor and a conjunctive agent are administered to a subject, the agents may be administered at the same time, but it is also possible that the conjunctive agent is first administered and then the KHK inhibitor is administered, or that the KHK is first administered and then the conjunctive agent is administered. When such an intermittent administration is employed, the time interval may vary depending on the active ingredient administered, the dosage form and the administration mode, and for example, when the conjunctive agent is first administered, the KHK-inhibitor may be administered within 1 minute to 3 days, preferably 10 minutes to 1 day, more preferably 15 minutes to 1 hour after the administration of the conjunctive agent. When the KHK inhibitor is first administered, for example, then the conjunctive agent may be administered within 1 minute to 1 day, preferably 10 minutes to 6 hours, more preferably 15 minutes to 1 hour after the administration of the KHK inhibitor.

It is understood that when referring to a KHK inhibitor and a conjunctive agent, it is meant a KHK inhibitor alone, a conjunctive agent alone, as a part of a composition, e.g., composition, which optionally includes one or more pharmaceutical carriers. It is also contemplated that more than one conjunctive agent may be administered to the subject if desired.

3. Polypeptides

KHK polypeptides according to an aspect of the present invention comprise at least 12, 15, 25, 50, 75, 100, 125, 150, 175, 200, 225, 250 or 265 contiguous amino acids selected from the amino acid sequence shown in SEQ ID NO: 2 and 4 (FIG. 6), or a biologically active variant thereof, as defined below. A KHK polypeptide of the invention therefore can be a portion of a KHK protein, a full-length KHK protein, or a fusion protein comprising all or a portion of KHK protein.

### 3.1 Biologically Active Variants

KHK polypeptide variants which are biologically active, i.e., confer an ability to phosphorylate fructose, also are considered KHK polypeptides for purposes of this application. Preferably, naturally or non-naturally occurring KHK polypeptide variants have amino acid sequences which are at least about 55, 60, 65, or 70, preferably about 75, 80, 85, 90, 96, 96, or 98% identical to the amino acid sequence shown in SEQ ID NO: 2 or a fragment thereof. Percent identity between a putative KHK polypeptide variant and an amino acid sequence of SEQ ID NO: 2 is determined using the Blast2 alignment program (Blosum62, Expect 10, standard genetic codes).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a KHK polypeptide can be found using computer programs well known in the art, such as DNASTAR software. Whether an amino acid change results in a biologically active KHK polypeptide can readily be determined by assaying for KHK activity, as described herein, for example.

### 3.2 Fusion Proteins

In some embodiments of the invention, it is useful to create fusion proteins. By way of example, fusion proteins are useful for generating antibodies against KHK polypeptide amino acid sequences and for use in various assay systems. For example, fusion proteins can be used to identify proteins which interact with portions of a KHK polypeptide. Protein affinity chromatography or library-based assays for protein-protein interactions, such as the yeast two-hybrid or phage display systems, can be used for this purpose. Such methods are well known in the art and also can be used as drug screens.

A KHK polypeptide fusion protein comprises two polypeptide segments fused together by means of a peptide bond. For example, the first polypeptide segment can comprise at least 12, 15, 25, 50, 75, 100, 125, 150, 175, 200, 225, or 250 contiguous amino acids of SEQ ID NO: 2 or of a biologically active variant, such as those described above. The first polypeptide segment also can comprise full-length KHK protein.

The second polypeptide segment can be a full-length protein or a protein fragment. Proteins commonly used in fusion protein construction include galactosidase, glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (H is) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the KHK polypeptide-encoding sequence and the heterologous protein sequence, so that the KHK polypeptide can be cleaved and purified away from the heterologous moiety.

Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

4. Polynucleotides

A KHK polynucleotide can be single- or double-stranded and comprises a coding sequence or the complement of a coding sequence for a KHK polypeptide. A coding sequence for KHK polypeptide of SEQ ID NO: 2 or 4 is shown in SEQ ID NO: 1 or 3, respectively see FIG. 6.

Degenerate nucleotide sequences encoding KHK polypeptides, as well as homologous nucleotide sequences which are at least about 50, 55, 60, 65, 60, preferably about 75, 90, 96, or 98% identical to the nucleotide sequence shown in SEQ ID NO: 1 also are KHK-like enzyme polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of KHK polynucleotides which encode biologically active KHK polypeptides also are KHK polynucleotides.

### 4.1 Identification of Polynucleotide Variants and Homologs

Variants and homologs of the KHK polynucleotides described above also are KHK polynucleotides. Typically, homologous KHK polynucleotide sequences can be identified by hybridization of candidate polynucleotides to known KHK polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% basepair mismatches.

Species homologs of the KHK polynucleotides disclosed herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Variants of KHK polynucleotides or polynucleotides of other species can therefore be identified by hybridizing a putative homologous KHK polynucleotide with a polynucleotide having a nucleotide sequence of SEQ ID NO: 1 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to KHK polynucleotides or their complements following stringent hybridization and/or wash conditions also are KHK polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a KHK polynucleotide having a nucleotide sequence shown in SEQ ID NO: 1 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

### 4.2 Preparation of Polynucleotides

A naturally occurring KHK polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated KHK polynucleotides. For example, restriction enzymes and probes can be used to isolate polynucleotide fragments which comprises KHK nucleotide sequences. Isolated polynucleotides are in preparations which are free or at least 70, 80, or 90% free of other molecules.

KHK DNA molecules can be made with standard molecular biology techniques, using KHK mRNA as a template. KHK DNA molecules can thereafter be replicated using molecular biology techniques known in the art and disclosed in manuals such as Sambrook et al. (1989). An amplification technique, such as PCR, can be used to obtain additional copies of polynucleotides of the invention. The inventors have successfully demonstrated this approach.

Alternatively, synthetic chemistry techniques can be used to synthesize KHK polynucleotides. The degeneracy of the genetic code allows alternate nucleotide sequences to be synthesized which will encode a KHK polypeptide having, for example, an amino acid sequence shown in SEQ ID NO: 2 or a biologically active variant thereof.

### 4.3 Expression of Polynucleotides

To express a KHK polynucleotide, the polynucleotide can be inserted into an expression vector which contains the necessary elements for the transcription and translation of the inserted coding sequence. Methods which are well known to those skilled in the art can be used to construct expression vectors containing sequences encoding KHK polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding a KHK enzyme polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those nontranslated regions of the vector enhancers, promoters, 5′ and 3′ untranslated regions which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a KHK polypeptide, vectors based on SV40 or EBV can be used with an appropriate selectable marker.

5. Host Cells

According to certain embodiments of the subject invention, a KHK polynucleotide will need to be inserted into a host cell, for expression, processing and/or screening. A host cell strain can be chosen for its ability to modulate the expression of the inserted sequences or to process the expressed KHK polypeptide in the desired fashion. Such modifications of the polypeptide include, but are not limited to, acetylation, carboxylation, glycosylation, phosphorylation, lipidation, and acylation. Posttranslational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; 10801 University Boulevard, Manassas, Va. 20110-2209) and can be chosen to ensure the correct modification and processing of the foreign protein.

Stable expression is preferred for long-term, high yield production of recombinant proteins. For example, cell lines which stably express KHK polypeptides can be transformed using expression vectors which can contain viral origins of replication and/or endogenous expression elements and a selectable marker gene on the same or on a separate vector. Following the introduction of the vector, cells can be allowed to grow for 12 days in an enriched medium before they are switched to a selective medium. The purpose of the selectable marker is to confer resistance to selection, and its presence allows growth and recovery of cells which successfully express the introduced KHK sequences. Resistant clones of stably transformed cells can be proliferated using tissue culture techniques appropriate to the cell type. See, for example, ANIMAL CELL CULTURE, R. I. Freshney, ed., 1986.

### 5.1 Detecting Expression

A variety of protocols for detecting and measuring the expression of a KHK polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a KHK polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 12111216, 1983).

### 5.2 Expression and Purification of Polypeptides

Host cells transformed with nucleotide sequences encoding KHK polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode KHK polypeptides can be designed to contain signal sequences which direct secretion of soluble KHK polypeptides through a prokaryotic or eukaryotic cell membrane or which direct the membrane insertion of membrane-bound KHK polypeptide.

6. Antibodies

Antibodies are referenced herein and various aspects of the subject invention utilize antibodies specific to KHK polypeptide(s). As described above, one example of an therapeutic agent may pertain to an antibody. Any type of antibody known in the art can be generated to bind specifically to an epitope of a KHK polypeptide. “Antibody” as used herein includes intact immunoglobulin molecules, as well as fragments thereof, such as Fab, F(ab′)₂, and Fv, which are capable of binding an epitope of a KHK polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a KHK polypeptide can be used therapeutically, as mentioned, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen. Antibodies useful for embodiments of the subject invention may be polyclonal, but are preferably monoclonal antibodies.

7. Ribozymes

Ribozymes may be one category of compounds useful as therapeutic agents for modulating KHK activity. Ribozymes are RNA molecules with catalytic activity. See, e.g., Cech, Science 236, 15321539; 1987; Cech, Ann. Rev. Biochem. 59, 543568; 1990, Cech, Curr. Opin. Struct. Biol. 2, 605609; 1992, Couture & Stinchcomb, Trends Genet. 12, 510515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

Accordingly, another aspect of the invention pertains to using the coding sequence of a KHK polynucleotide to generate ribozymes which will specifically bind to mRNA transcribed from the KHK polynucleotide. Methods of designing and constructing ribozymes which can cleave other RNA molecules in trans in a highly sequence specific manner have been developed and described in the art (see Haseloff et al. Nature 334, 585591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a KHK RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate KHK RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease KHK expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, the entirety of which is incorporated by reference, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein.

8. Interfering Molecules

KHK can be inhibited by a number of means including silencing via miRNA, shRNA, or sRNA, for example, directed to a portion of the sequence described at the genbank accession numbers provided above. sRNA molecules can be prepared against a portion of SEQ. ID. Nos 1 and 3 according to the techniques provided in U.S. Patent Publication 20060110440 and used as therapeutic compounds. shRNA constructs are typically made from one of three possible methods; (i) annealed complementary oligonucleotides, (ii) promoter based PCR or (iii) primer extension. See Design and cloning strategies for constructing shRNA expression vectors, Glen J McIntyre, Gregory C FanningBMC Biotechnology 2006, 6:1 (5 Jan. 2006).

For background information on the preparation of miRNA molecules, see e.g. U.S. patent applications 20110020816, 2007/0099196; 2007/0099193; 2007/0009915; 2006/0130176; 2005/0277139; 2005/0075492; and 2004/0053411, the disclosures of which are hereby incorporated by reference herein. See also, U.S. Pat. Nos. 7,056,704 and 7,078,196 (preparation of miRNA molecules), incorporated by reference herein. Synthetic miRNAs are described in Vatolin, et al 2006 J Mol Biol 358, 983-6 and Tsuda, et al 2005 Int J Oncol 27, 1299-306, incorporated by reference herein.

It is within the scope of aspects of the present invention to provide agents to silence KHK (KHK-C or KHK-A and KHK-C) genes to achieve a therapeutic effect using interfering molecules. In certain embodiments, silencing of human KHK genes should be based on either or both of the sequences of the KHK enzymes mentioned above.

9. KHK Inhibitor Compounds

To document that small molecule compounds can be generated to inhibit KHK-C specifically, the present inventors conducted a virtual screen (computational docking experiment) of the crystal structure of KHK and identified compounds from the ZINC database, which had favorable docking scores and demonstrated complementary interactions with the protein based on a follow-up visual inspection of the proposed binding modes. Shown in Table 1 are several compounds that could preferentially inhibit KHK over KHK-A. For example, (Z)-3-(methylthio)-1-phenyl-N′-(((4-(trifluoromethoxy)phenyl)carbamoyl)oxy)-1H-pyrazole-4-carboximidamide, [1 in Table 1 below], shows 25.6% inhibition of KHKC at 10 uM and 7.0% inhibition of KHKA at 10 uM. 5-amino-3-(methylthio)-1-phenyl-1H-pyrazole-4-carbonitrile, [2 in Table 1 below], shows 16.8% inhibition of KHKC at 100 uM and 29.4% inhibition of KHKA at 100 uM. 2-(3-(methylthio)-1-phenyl-1H-pyrazol-4-yl)-4-phenylthiazole, [3 in Table 1 below], shows 19.9% inhibition of KHKC at 10 uM.

TABLE 1
KHK Inhibitors:

| | | | KHKC % | KHKC % | KHKA % | KHKA % | |
|---|---|---|---|---|---|---|---|
| | Cpd | | Inhibition | Inhibition | Inhibition | Inhibition | |
| Structure | No. | MW | (10 UM) | (100 uM) | (10 UM) | (100 uM) | IUPAC NAME |
| 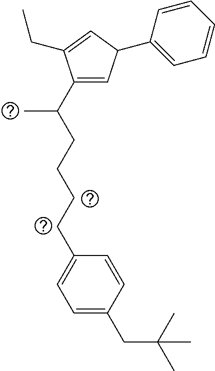 [SMILES: CCC1=CC(c2ccccc2)C=C1C(C)CCCCc1ccc(CC(C)(C)C)cc1] [symbol] | 1 | 451.4 | 25.6 | | 7.0 | | (Z)-3-(methylthio)-1-phenyl-N′-(((4- (trifluoromethoxy)phenyl)carbamoyl)oxy)- 1H-pyrazole-4-carboximidamide |
| 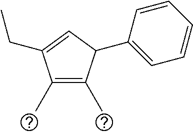 [symbol] | 2 | 230.3 | 7.8 | 16.8 | 5.7 | 29.4 | 5-amino-3-(methylthio)-1-phenyl-1H- pyrazole-4-carbonitrile |
| 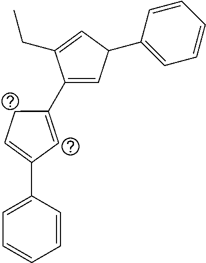 [SMILES: CCC1=CC(c2ccccc2)C=C1C1=CC(c2ccccc2)=CC1] [symbol] | 3 | 349.5 | 19.9 | | | | 2-(3-(methylthio)-1-phenyl-1H-pyrazol- 4-yl)-4-phenylthiazole |

[symbol]

indicates data missing or illegible when filed

In accordance with one aspect of the present invention, there is thus provided a method for inhibiting KHK activity in a subject. The method comprises administering to the subject an effective amount of a compound selected from the group consisting of:

(Z)-3-(methylthio)-1-phenyl-N′-(((4-(trifluoromethoxy)phenyl)carbamoyl)oxy)-1H-pyrazole-4-carboximidamide;

5-amino-3-(methylthio)-1-phenyl-1H-pyrazole-4-carbonitrile;

2-(3-(methylthio)-1-phenyl-1H-pyrazol-4-yl)-4-phenylthiazole; and

combinations thereof.

In accordance with another aspect of the present invention, there is provided a composition, e.g., a pharmaceutical composition, comprising a KHK inhibitor, wherein the KHK inhibitor comprises a compound selected from the group consisting of:

(Z)-3-(methylthio)-1-phenyl-N′-(((4-(trifluoromethoxy)phenyl)carbamoyl)oxy)-1H-pyrazole-4-carboximidamide;

5-amino-3-(methylthio)-1-phenyl-1H-pyrazole-4-carbonitrile;

2-(3-(methylthio)-1-phenyl-1H-pyrazol-4-yl)-4-phenylthiazole; and

combinations thereof.

Other fructokinase inhibitors (nonspecific) include 4 hydroxymercuric benzoic acid. Further exemplary KHK inhibitor compounds and methods for their synthesis are set forth at:
Gibbs, AC, Abad, M C, Zhang, X, Toungue, BA, Lewandowski, F A, Struble, G T, Sun W, Sui Z, Kuo L. Electron Density Guided Fragment-Based Lead Discovery of Ketohexokinase Inhibitors. J. Med. Chem. 2010, 53, 7979-7991, the entirety of which is hereby incorporated by reference herein.
Maryanoff, BE, O'Neill, J.C., McComsey, D F, Yabut, S C, Luci, D K, Jordan, Jr., AD, Masucci, JA, Jones, W J, Abad, M C, Gibbs, AC, and Petrounia, I. Inhibitors of Ketohexokinase: Discovery of Pyrimidinopyrimidines with Specific Substitution that Complements the ATP-Binding Site. Dx.doi.org/10.1021/ml200070g: ACS Med. Chem. Lett. XXXX, XXX, 000-000, the entirety of which is hereby incorporated by reference herein.
X. Zhang et al. Optimization of a pyrazole hit from FBDD into a novel series of indazoles as ketohexokinase inhibitors. Bioorg. Med. Chem. Lett. 21 (2011) 4762-4767, the entirety of which is hereby incorporated by reference herein.

### X. FRUCTANASE INHIBITORS

Suitable fructanase inhibitors for use in the present invention include, but are not limited to, iodoacetic acid. The fructanase inhibitor may also include one or more of a ribozyme, an interfering molecule, a peptide, a small molecule, or an antibody targeted to fructanase. In one embodiment, fructikinase can be inhibited by silencing expression of fructanase via miRNA, shRNA, or sRNA, for example, directed to a portion of the sequence described at the relevant accession number, e.g., genbank accession number F8LQJ7.

### XI. PHARMACEUTICAL COMPOSITIONS

The fructokinase and/or fructanase inhibitors described herein may be formulated as a pharmaceutical composition suitable for administration to a subject. Such compositions typically comprise the first angiogenesis inhibitor, e.g., sRNA molecule, and a pharmaceutically acceptable carrier. Exemplary pharmaceutically acceptable carriers include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

The composition may be delivered to the subject via any suitable route of administration such that the composition (comprising at least the first angiogenesis inhibitor) may perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another. In one embodiment, the intended target of the first angiogenesis inhibitor is the blood vessels of the subject's circulatory system. Accordingly, in one embodiment, the composition is delivered intravenously.

### Examples

Shown below are evidence that fructose can accelerate the development of food allergy in mice. In experiment 1, allergy-susceptible mice (lacking the toll-like receptor 4) were given a conditioning protocol in which they receive weekly gavage of peanuts (10 mg) with cholera toxin (20 μg) for 4 weeks followed by a challenge of peanut proteins (50 mg) at week 5. Group 2 received peanut proteins and fructose (30% in water) and Group 3 received peanuts plus cholera toxin (an enterotoxin) plus fructose. Allergic response was assessed by symptom score (0-5) and temperature (with degree of falling temperature being consistent with severity of anaphylaxis). In addition, serum IgE levels to peanut antigens were measured at week 6. As shown in Table 2, the addition of fructose to group 3 was associated with numerically worse symptoms and a greater fall in temperature compared to group 1.

TABLE 2
Experiment 1

| | Peanut | Symptom | Temperature |
|---|---|---|---|
| Results: | IgE (ng/ml) | score | drop (C. °) |
| Group 1: | 340 | 3 | 4.1 |
| (Peanuts + CT) | 430 | 3 | 4.4 |
| | 205 | 3 | 3.3 |
| Group 2: | 26 | 0 | 0 |
| (Peanuts + fructose) | 105 | 0 | 0 |
| | 50 | 2 | 3 |
| | 0 | 0 | 0 |
| Group 3: | 250 | 4 | 6.4 |
| (Peanuts + Fructose + CT) | 390 | 3 | 2.6 |
| | 430 | 4 | 7.2 |

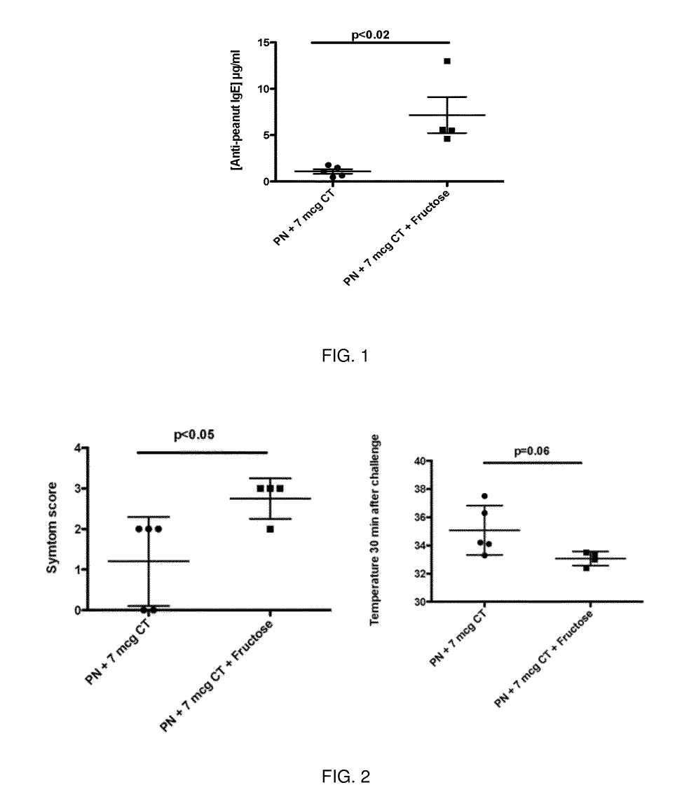

In experiment 2, we performed a similar experiment in which we compared peanuts plus cholera toxin with and without added fructose. Fructose (30%) was started for two weeks before sensitization was started. At week 5 mice were challenged with peanut proteins (50 mg) and assessed similarly as experiment 1. As shown in FIG. 1, peanut IgE levels were higher in the group receiving fructose consistent with fructose increasing the risk for peanut IgE response (p<0.02 by Mann Whitney test).

In addition, as shown in FIG. 2, the mice receiving peanuts plus cholera toxin plus fructose showed a worse symptom score (P<0.05) and fall in temperature (P=0.06) compared to mice receiving peanuts with low dose cholera toxin alone. These studies document that fructose accelerates the development of IgE mediated food allergy in mice. These data show that fructose increases the risk for severe allergic reaction to peanuts.

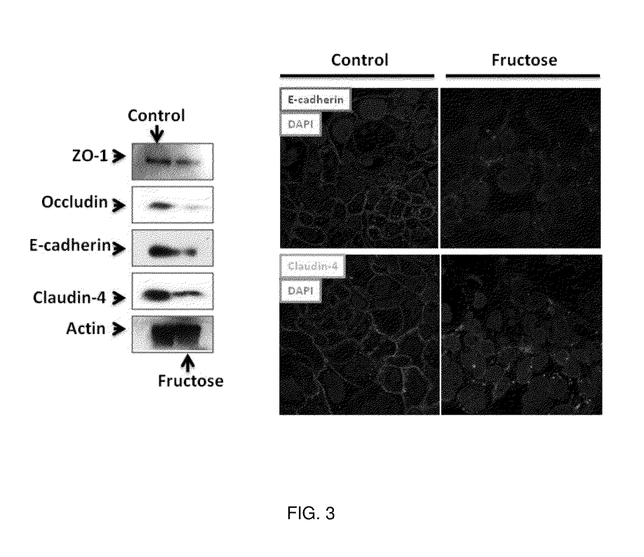

As set forth in FIG. 3, in vitro studies employing human intestinal epithelial cells (CaCo-2) revealed that exposure of these cells to 5 mM fructose markedly decreased the expression of genes involved in the maintenance of cell polarity. Specifically, the expression of e-cadherin, a marker of epithelial cells, is dramatically down-regulated in cells exposed to fructose for 96 hours. Consistent with this finding the expression of both transmembrane (claudin-4) and scaffolding (ZO-1 and occluding) proteins at the tight junctions is substantially down-regulated (left). Confocal analysis of CaCo-2 cells revealed that fructose not only down-regulates the protein expression of these genes involved in cell polarity and paracellular flux (claudin-4 is known to decrease paracellular flux of ions and is in fact the target for the cholera toxin), but also translocates them intracellularly from the basolateral membrane domain (right).

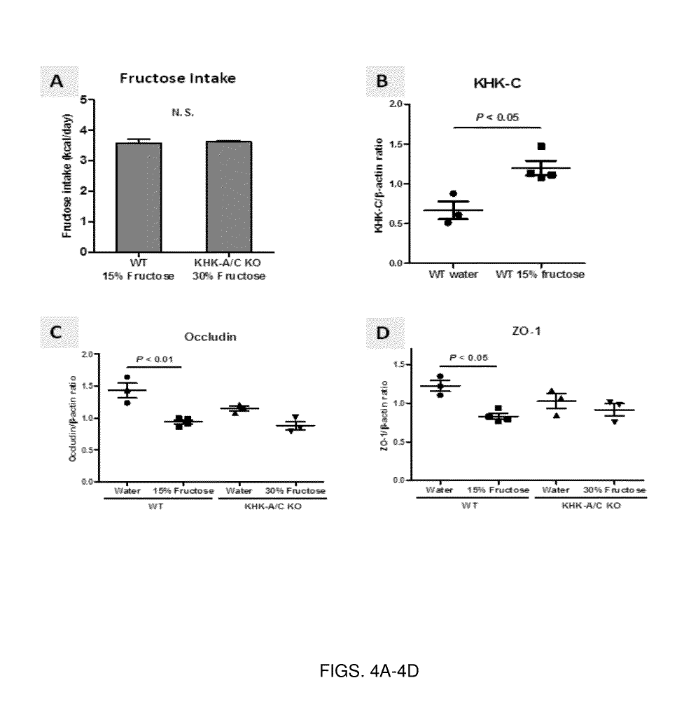

Studies were also performed to show that fructose causes an alteration in intestinal permeability as a consequence of fructokinase. In the first set of studies, three-month-old male wild type (WT) mice or fructokinase A/C knockout mice (KHK-A/C KO) (obtained from David Bonthron, Leeds, UK)(72) were administered fructose with normal chow for 3 weeks. Two additional groups of WT mice and KHK-A/C KO mice were given normal chow with tap water that did not contain fructose as a control. Due to the different preference for fructose water between WT mice and KHK-A/C KO mice, mice were given 15% or 30% of fructose water, respectively. Both WT mice and KHK-A/C KO mice drank the same amount of fructose (FIG. 4A). For each group, the duodenum was removed after 20 hours of fasting of normal chow and the lining scraped and RNA extracted. Quantitative real time PCR was performed for fructokinase C (KHK-C, FIG. 4B), and tight junction genes occludin (FIG. 4C) and ZO-1 (FIG. 4D) using actin as an internal control. As shown, WT mice fed fructose show an upregulation of KHK mRNA expression in association with a significant decrease in occludin and ZO-1 mRNA. These studies suggest fructose is increasing intestinal permeability. The observation that this does not occur in fructokinase KO mice suggests that the intestinal permeability is mediated by fructokinase.

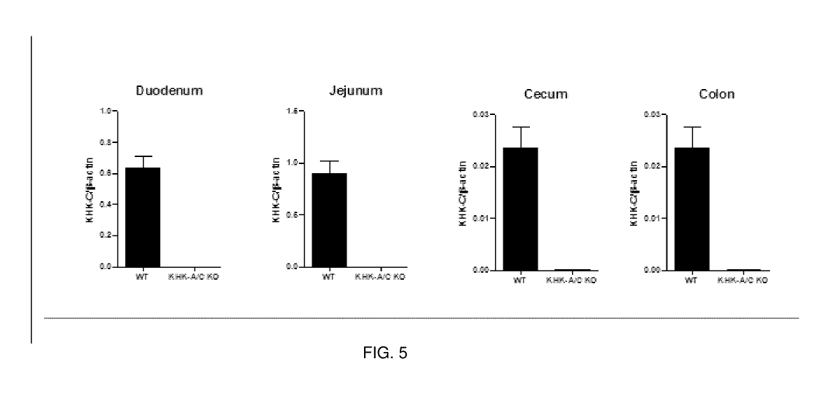

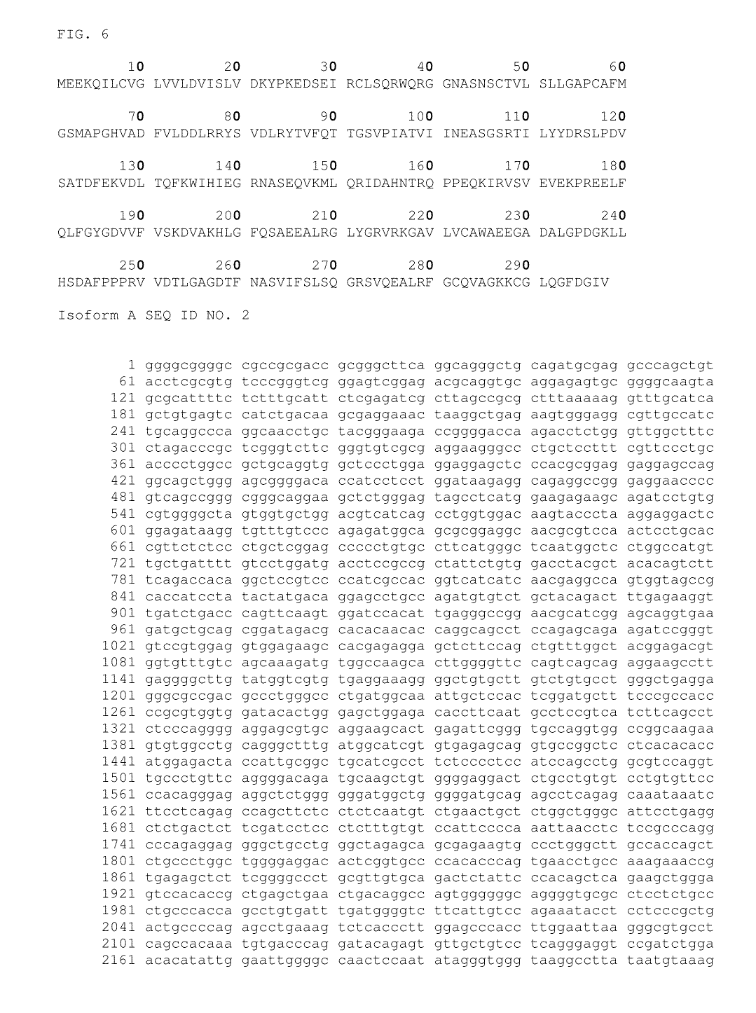

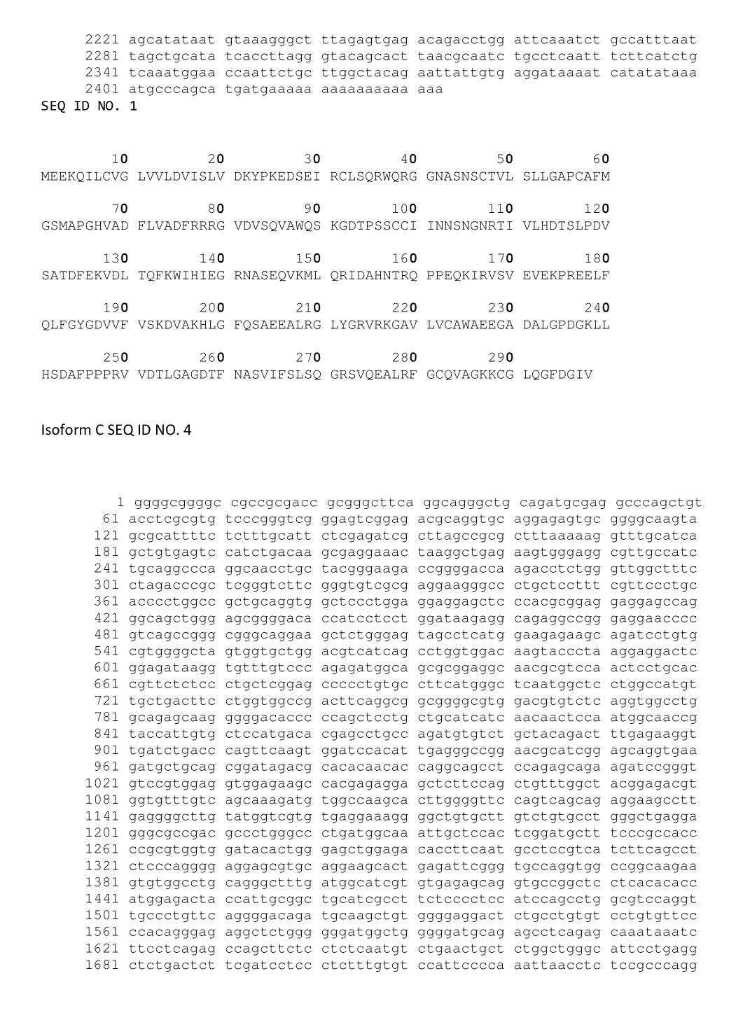

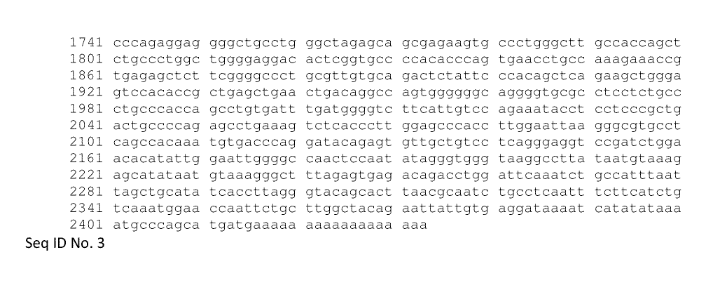

In addition, as shown in FIG. 5, separate studies showed that fructokinase C (KHK-C) is expressed throughout the intestinal tract, including the duodenum, jejunum, cecum and colon whereas it is not expressed in mice in which both fructokinase C and A have been knocked out (KHK-A/C KO).

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

### REFERENCES

1. Asipu A, Hayward B E, O'Reilly J, Bonthron D T. Properties of normal and mutant recombinant human ketohexokinases and implications for the pathogenesis of essential fructosuria. Diabetes 2003; 52:2426-32.
2. Diggle C P, Shires M, McRae C, et al. Both isoforms of ketohexokinase are dispensable for normal growth and development. Physiol Genomics 2010; 42A:235-43.
3. Diggle C P, Shires M, Leitch D, et al. Ketohexokinase: expression and localization of the principal fructose-metabolizing enzyme. J Histochem Cytochem 2009; 57:763-74.
4. Cirillo P, Gersch M S, Mu W, et al. Ketohexokinase-dependent metabolism of fructose induces proinflammatory mediators in proximal tubular cells. J Am Soc Nephrol 2009; 20:545-53.
5. Lanaspa M, Sanchez-Lozada L G, Kanbay M, et al. Activation of purine metabolism pathway and generation of uric acid contributes to the development of fatty liver. Nature Med 2011;in revision.
6. van den Berghe G, Bronfman M, Vanneste R, Hers H G. The mechanism of adenosine triphosphate depletion in the liver after a load of fructose. A kinetic study of liver adenylate deaminase. The Biochemical journal 1977; 162:601-9.
7. Maenpaa P H, Raivio K O, Kekomaki M P. Liver adenine nucleotides: fructose-induced depletion and its effect on protein synthesis. Science (New York, N.Y. 1968; 161:1253-4.
8. Bergheim I, Weber S, Vos M, et al. Antibiotics protect against fructose-induced hepatic lipid accumulation in mice: role of endotoxin. J Hepatol 2008; 48:983-92.
9. Johnson R J, Perez-Pozo S E, Sautin Y Y, et al. Hypothesis: could excessive fructose intake and uric acid cause type 2 diabetes? Endocr Rev 2009; 30:96-116.
10. Venter C, Arshad S H. Epidemiology of food allergy. Pediatr Clin North Am 2011; 58:327-49, ix.
11. Sicherer S H. Epidemiology of food allergy. J Allergy Clin Immunol 2011; 127:594-602.
12. Walker W A. Pathophysiology of intestinal uptake and absorption of antigens in food allergy. Ann Allergy 1987; 59:7-16.
13. Perrier C, Corthesy B. Gut permeability and food allergies. Clin Exp Allergy 2011; 41:20-8.
14. Strait R T, Mahler A, Hogan S, Khodoun M, Shibuya A, Finkelman F D. Ingested allergens must be absorbed systemically to induce systemic anaphylaxis. J Allergy Clin Immunol 2011; 127:982-9 e1.
15. Sicherer S H, Sampson H A. Food allergy: recent advances in pathophysiology and treatment. Annu Rev Med 2009; 60:261-77.
16. Garcia-Manzanares A, Lucendo A J. Nutritional and dietary aspects of celiac disease. Nutr Clin Pract 2011; 26:163-73.
17. Lionetti E, Catassi C. New clues in celiac disease epidemiology, pathogenesis, clinical manifestations, and treatment. Int Rev Immunol 2011; 30:219-31.
18. Vogelsang H, Schwarzenhofer M, Oberhuber G. Changes in gastrointestinal permeability in celiac disease. Dig Dis 1998; 16:333-6.
19. Stenhammar L, Stromberg S. Intestinal permeability to lactulose/L-rhamnose in children with celiac disease and other gastrointestinal disorders. J Pediatr Gastroenterol Nutr 1988; 7:304-6.
20. Smecuol E, Bai J C, Vazquez H, et al. Gastrointestinal permeability in celiac disease. Gastroenterology 1997; 112:1129-36.
21. Cox M A, Lewis K O, Cooper B T. Sucrosemia in untreated celiac disease: a potential screening test. Dig Dis Sci 1998; 43:1096-101.
22. Ludvigsson J F, Montgomery S M, Ekbom A, Brandt L, Granath F. Small-intestinal histopathology and mortality risk in celiac disease. JAMA 2009; 302:1171-8.
23. Ciacci C, Gennarelli D, Esposito G, Tortora R, Salvatore F, Sacchetti L. Hereditary fructose intolerance and celiac disease: a novel genetic association. Clin Gastroenterol Hepatol 2006; 4:635-8.
24. Diosdado B, Wapenaar M C, Franke L, et al. A microarray screen for novel candidate genes in coeliac disease pathogenesis. Gut 2004; 53:944-51.
25. Boda M, Nemeth I, Boda D. The caffeine metabolic ratio as an index of xanthine oxidase activity in clinically active and silent celiac patients. J Pediatr Gastroenterol Nutr 1999; 29:546-50.
26. Reif S, Klein I, Lubin F, Farbstein M, Hallak A, Gilat T. Pre-illness dietary factors in inflammatory bowel disease. Gut 1997; 40:754-60.
27. Mahmud N, Weir D G. The urban diet and Crohn's disease: is there a relationship? Eur J Gastroenterol Hepatol 2001; 13:93-5.
28. Jarnerot G, Jarnmark I, Nilsson K. Consumption of refined sugar by patients with Crohn's disease, ulcerative colitis, or irritable bowel syndrome. Scand J Gastroenterol 1983; 18:999-1002.
29. Riordan A M, Ruxton C H, Hunter J O. A review of associations between Crohn's disease and consumption of sugars. European journal of clinical nutrition 1998; 52:229-38.
30. Carbonnel F, Boutron M C. Ulcerative colitis: is it in the diet? Gut 2009; 58:1577-9.
31. Carbonnel F, Jantchou P, Monnet E, Cosnes J. Environmental risk factors in Crohn's disease and ulcerative colitis: an update. Gastroenterol Clin Biol 2009; 33 Suppl 3:S145-57.
32. Zeissig S, Burgel N, Gunzel D, et al. Changes in expression and distribution of claudin 2, 5 and 8 lead to discontinuous tight junctions and barrier dysfunction in active Crohn's disease. Gut 2007; 56:61-72.
33. Irvine E J, Marshall J K. Increased intestinal permeability precedes the onset of Crohn's disease in a subject with familial risk. Gastroenterology 2000; 119:1740-4.
34. Khor B, Gardet A, Xavier R J. Genetics and pathogenesis of inflammatory bowel disease. Nature 2011; 474:307-17.
35. Vogelsang H. Do changes in intestinal permeability predict disease relapse in Crohn's disease? Inflamm Bowel Dis 2008; 14 Suppl 2:S162-3.
36. Shen L, Su L, Turner J R. Mechanisms and functional implications of intestinal barrier defects. Dig Dis 2009; 27:443-9.
37. Davin J C, Forget P, Mahieu P R. Increased intestinal permeability to (51 Cr) EDTA is correlated with IgA immune complex-plasma levels in children with IgA-associated nephropathies. Acta Paediatr Scand 1988; 77:118-24.
38. Kovacs T, Kun L, Schmelczer M, Wagner L, Davin J C, Nagy J. Do intestinal hyperpermeability and the related food antigens play a role in the progression of IgA nephropathy? I. Study of intestinal permeability. American journal of nephrology 1996; 16:500-5.
39. Coppo R, Amore A, Roccatello D, et al. IgA antibodies to dietary antigens and lectin-binding IgA in sera from Italian, Australian, and Japanese IgA nephropathy patients. Am J Kidney Dis 1991; 17:480-7.
40. Nagy J, Scott H, Brandtzaeg P. Antibodies to dietary antigens in IgA nephropathy. Clin Nephrol 1988; 29:275-9.
41. Feehally J. IgA nephropathy—a disorder of IgA production? Qjm 1997; 90:387-90.
42. Wakai K, Nakai S, Matsuo S, et al. Risk factors for IgA nephropathy: a case-control study with incident cases in Japan. Nephron 2002; 90:16-23.
43. Waldhoff D, Saint-Paul U, Furch B. Value of Fruits and Seeds from the Floodplain Forests of Central Amazonia as food resource for fish. Ecotropica 1996; 2:143-6.
44. Pollitt C C. Equine Laminitis. Australia; 2001.
45. Carter R A, Treiber K H, Geor R J, Douglass L, Harris P A. Prediction of incipient pasture-associated laminitis from hyperinsulinaemia, hyperleptinaemia and generalised and localised obesity in a cohort of ponies. Equine Vet J 2009; 41:171-8.
46. Bailey S R, Habershon-Butcher J L, Ransom K J, Elliott J, Menzies-Gow N J. Hypertension and insulin resistance in a mixed-breed population of ponies predisposed to laminitis. Am J Vet Res 2008; 69:122-9.
47. van Eps A W, Pollitt C C. Equine laminitis induced with oligofructose. Equine Vet J 2006; 38:203-8.
48. Andersen R, Sorensen A. An enzymatic method for the determination of fructans in foods and food products. Eur Food Res Tech 1999; 210.
49. McMeniman N P. Nutrition of Grazing Broodmares, Their Foals and Young horses. In. Barton Australia: Rural Industries Research and Development Corporation; 2000.
50. Nourian A R, Asplin K E, McGowan C M, Sillence M N, Pollitt C C. Equine laminitis: ultrastructural lesions detected in ponies following hyperinsulinaemia. Equine Vet J 2009; 41:671-7.
51. Milinovich G J, Burrell P C, Pollitt C C, et al. Microbial ecology of the equine hindgut during oligofructose-induced laminitis. ISME J 2008; 2:1089-100.
52. Pollitt C C. Equine laminitis. Clin Tech Equine Pract 2004; 3:34-44.
53. Singh P, Gill P K. Production of inulinases: Recent advances. Food Technol Biotechnol 2006; 44:151-62.
54. De Fombelle A, Varloud M, Goachet A-G, et al. Characterization of the microbial and biochemical profile of the different segments of the digestive tract in horses given two distinct diets. J Animal Sci 2003; 77:293-304.
55. Coenen M, Mosseler A, Vervuert I. Fermentative gases in breath indicate that inulin and starch start to be degraded by microbial fermentation in the stomach and small intestine of the horse in contrast to pectin and cellulose. The Journal of nutrition 2006; 136:2108 S-10S.
56. Nakagawa T, Hu H, Zharikov S, et al. A causal role for uric acid in fructose-induced metabolic syndrome. American journal of physiology 2006; 290:F625-31.
57. Ley R E, Turnbaugh P J, Klein S, Gordon J I. Microbial ecology: human gut microbes associated with obesity. Nature 2006; 444:1022-3.
58. Ley R E, Backhed F, Turnbaugh P, Lozupone C A, Knight R D, Gordon J I. Obesity alters gut microbial ecology. Proceedings of the National Academy of Sciences of the United States of America 2005; 102:11070-5.
59. Turnbaugh P J, Ley R E, Mahowald M A, Magrini V, Mardis E R, Gordon J I. An obesity-associated gut microbiome with increased capacity for energy harvest. Nature 2006; 444:1027-31.
60. Turnbaugh P J, Backhed F, Fulton L, Gordon J I. Diet-induced obesity is linked to marked but reversible alterations in the mouse distal gut microbiome. Cell Host Microbe 2008; 3:213-23.
61. Backhed F, Manchester J K, Semenkovich C F, Gordon J I. Mechanisms underlying the resistance to diet-induced obesity in germ-free mice. Proceedings of the National Academy of Sciences of the United States of America 2007; 104:979-84.
62. Muccioli G G, Naslain D, Backhed F, et al. The endocannabinoid system links gut microbiota to adipogenesis. Mol Syst Biol 2010; 6:392.
63. Strader A D. Ileal transposition provides insight into the effectiveness of gastric bypass surgery. Physiology & behavior 2006; 88:277-82.
64. Rubino F, Marescaux J. Effect of duodenal-jejunal exclusion in a non-obese animal model of type 2 diabetes: a new perspective for an old disease. Ann Surg 2004; 239:1-11.
65. Mingrone G, Castagneto-Gissey L. Mechanisms of early improvement/resolution of type 2 diabetes after bariatric surgery. Diabetes Metab 2009; 35:518-23.
66. Cummings D E. Endocrine mechanisms mediating remission of diabetes after gastric bypass surgery. Int J Obes (Lond) 2009; 33 Suppl 1:S33-40.
67. Bose M, Olivan B, Teixeira J, Pi-Sunyer F X, Laferrere B. Do Incretins play a role in the remission of type 2 diabetes after gastric bypass surgery: What are the evidence? Obes Surg 2009; 19:217-29.
68. Andreelli F, Amouyal C, Magnan C, Mithieux G. What can bariatric surgery teach us about the pathophysiology of type 2 diabetes? Diabetes Metab 2009; 35:499-507.

All references and documents cited herein are incorporated herein in their entirety to the extent not inconsistent with the teachings herein.

1-8. (canceled)

9. A method for the treatment or prevention of a food allergy in a subject comprising administering to the subject an effective amount of a KHK inhibitor.

10. The method of claim 9, wherein the food allergy comprises an IgE-mediated food allergy.

11. The method of claim 9, wherein the food allergy comprises an allergy to a member selected from the group consisting of milk, eggs, peanuts, tree nuts, fish, and shellfish.

12-14. (canceled)

15. A method for treating or preventing an Inflammatory Bowel Disease in a subject comprising administering to the subject an effective amount of a KHK inhibitor.

16. The method of claim 15, where in the Inflammatory Bowel Disease comprises Chrohn's disease or ulcerative colitis.

17-41. (canceled)

42. A method of treating a fructose-associated intestinal disorder or disease in a subject, said method comprising administering to the subject an effective amount of a fructokinase inhibitor, a fructanase inhibitor, or antibiotic specific to fructanase-producing bacteria, or a combination thereof.

43. The method of claim 42, wherein said fructose-associated intestinal disease or disorder is irritable bowel syndrome.

44. The method of claim 42, wherein said fructose-associated intestinal disease or disorder is celiac disease.

45. The method of claim 42, wherein said fructose-associated intestinal disease or disorder is founder disease.

46. The method of claim 42, wherein said fructose-associated intestinal disease or disorder is inflammatory bowel disease.

47. The method of claim 42, wherein said fructose-associated intestinal disorder is gut bacteria-induced obesity.

48. The method of claim 42, wherein said administering comprises administering to the subject an effective amount of a fructokinase inhibitor.

49. The method of claim 42, wherein said administering comprises administering to the subject an effective amount of a fructanase inhibitor.

50. The method of claim 42, wherein said administering comprises administering to the subject an effective amount of an antibiotic specific to fructanase-producing bacteria.

51. The method of claim 42, wherein said fructose-associated intestinal disorder is equine metabolic syndrome and the subject is an equine animal.

52. The method of claim 51, wherein administering comprises administering to the equine animal an effective amount of an antibiotic effective to reduce an amount of fructanase-producing bacteria in the subject.

53. The method of claim 52, wherein said antibiotic is vancomycin.


SEQUENCE LISTING (4 sequences)

SEQ ID NO: 1 - DNA, 2433 bp, Homo sapiens
ggggcggggccgccgcgaccgcgggcttcaggcagggctgcagatgcgaggcccagctgtacctcgcgtgtcccgggtcgggagtcggagacgcaggtgcaggagagtgcggggcaagtagcgcattttctctttgcattctcgagatcgcttagccgcgctttaaaaaggtttgcatcagctgtgagtccatctgacaagcgaggaaactaaggctgagaagtgggaggcgttgccatctgcaggcccaggcaacctgctacgggaagaccggggaccaagacctctgggttggctttcctagacccgctcgggtcttcgggtgtcgcgaggaagggccctgctcctttcgttccctgcacccctggccgctgcaggtggctccctggaggaggagctcccacgcggaggaggagccagggcagctgggagcggggacaccatcctcctggataagaggcagaggccgggaggaaccccgtcagccgggcgggcaggaagctctgggagtagcctcatggaagagaagcagatcctgtgcgtggggctagtggtgctggacgtcatcagcctggtggacaagtaccctaaggaggactcggagataaggtgtttgtcccagagatggcagcgcggaggcaacgcgtccaactcctgcaccgttctctccctgctcggagccccctgtgccttcatgggctcaatggctcctggccatgttgctgattttgtcctggatgacctccgccgctattctgtggacctacgctacacagtctttcagaccacaggctccgtccccatcgccacggtcatcatcaacgaggccagtggtagccgcaccatcctatactatgacaggagcctgccagatgtgtctgctacagactttgagaaggttgatctgacccagttcaagtggatccacattgagggccggaacgcatcggagcaggtgaagatgctgcagcggatagacgcacacaacaccaggcagcctccagagcagaagatccgggtgtccgtggaggtggagaagccacgagaggagctcttccagctgtttggctacggagacgtggtgtttgtcagcaaagatgtggccaagcacttggggttccagtcagcagaggaagccttgaggggcttgtatggtcgtgtgaggaaaggggctgtgcttgtctgtgcctgggctgaggagggcgccgacgccctgggccctgatggcaaattgctccactcggatgctttcccgccaccccgcgtggtggatacactgggagctggagacaccttcaatgcctccgtcatcttcagcctctcccaggggaggagcgtgcaggaagcactgagattcgggtgccaggtggccggcaagaagtgtggcctgcagggctttgatggcatcgtgtgagagcaggtgccggctcctcacacaccatggagactaccattgcggctgcatcgccttctcccctccatccagcctggcgtccaggttgccctgttcaggggacagatgcaagctgtggggaggactctgcctgtgtcctgtgttccccacagggagaggctctggggggatggctgggggatgcagagcctcagagcaaataaatcttcctcagagccagcttctcctctcaatgtctgaactgctctggctgggcattcctgaggctctgactcttcgatcctccctctttgtgtccattccccaaattaacctctccgcccaggcccagaggaggggctgcctgggctagagcagcgagaagtgccctgggcttgccaccagctctgccctggctggggaggacactcggtgccccacacccagtgaacctgccaaagaaaccgtgagagctcttcggggccctgcgttgtgcagactctattcccacagctcagaagctgggagtccacaccgctgagctgaactgacaggccagtggggggcaggggtgcgcctcctctgccctgcccaccagcctgtgatttgatggggtcttcattgtccagaaatacctcctcccgctgactgccccagagcctgaaagtctcacccttggagcccaccttggaattaagggcgtgcctcagccacaaatgtgacccaggatacagagtgttgctgtcctcagggaggtccgatctggaacacatattggaattggggccaactccaatatagggtgggtaaggccttataatgtaaagagcatataatgtaaagggctttagagtgagacagacctggattcaaatctgccatttaattagctgcatatcaccttagggtacagcacttaacgcaatctgcctcaatttcttcatctgtcaaatggaaccaattctgcttggctacagaattattgtgaggataaaatcatatataaaatgcccagcatgatgaaaaaaaaaaaaaaaaaa

SEQ ID NO: 2 - protein, 298 aa, Homo sapiens
MEEKQILCVGLVVLDVISLVDKYPKEDSEIRCLSQRWQRGGNASNSCTVLSLLGAPCAFMGSMAPGHVADFVLDDLRRYSVDLRYTVFQTTGSVPIATVIINEASGSRTILYYDRSLPDVSATDFEKVDLTQFKWIHIEGRNASEQVKMLQRIDAHNTRQPPEQKIRVSVEVEKPREELFQLFGYGDVVFVSKDVAKHLGFQSAEEALRGLYGRVRKGAVLVCAWAEEGADALGPDGKLLHSDAFPPPRVVDTLGAGDTFNASVIFSLSQGRSVQEALRFGCQVAGKKCGLQGFDGIV

SEQ ID NO: 3 - DNA, 2433 bp, Homo sapiens
ggggcggggccgccgcgaccgcgggcttcaggcagggctgcagatgcgaggcccagctgtacctcgcgtgtcccgggtcgggagtcggagacgcaggtgcaggagagtgcggggcaagtagcgcattttctctttgcattctcgagatcgcttagccgcgctttaaaaaggtttgcatcagctgtgagtccatctgacaagcgaggaaactaaggctgagaagtgggaggcgttgccatctgcaggcccaggcaacctgctacgggaagaccggggaccaagacctctgggttggctttcctagacccgctcgggtcttcgggtgtcgcgaggaagggccctgctcctttcgttccctgcacccctggccgctgcaggtggctccctggaggaggagctcccacgcggaggaggagccagggcagctgggagcggggacaccatcctcctggataagaggcagaggccgggaggaaccccgtcagccgggcgggcaggaagctctgggagtagcctcatggaagagaagcagatcctgtgcgtggggctagtggtgctggacgtcatcagcctggtggacaagtaccctaaggaggactcggagataaggtgtttgtcccagagatggcagcgcggaggcaacgcgtccaactcctgcaccgttctctccctgctcggagccccctgtgccttcatgggctcaatggctcctggccatgttgctgacttcctggtggccgacttcaggcggcggggcgtggacgtgtctcaggtggcctggcagagcaagggggacacccccagctcctgctgcatcatcaacaactccaatggcaaccgtaccattgtgctccatgacacgagcctgccagatgtgtctgctacagactttgagaaggttgatctgacccagttcaagtggatccacattgagggccggaacgcatcggagcaggtgaagatgctgcagcggatagacgcacacaacaccaggcagcctccagagcagaagatccgggtgtccgtggaggtggagaagccacgagaggagctcttccagctgtttggctacggagacgtggtgtttgtcagcaaagatgtggccaagcacttggggttccagtcagcagaggaagccttgaggggcttgtatggtcgtgtgaggaaaggggctgtgcttgtctgtgcctgggctgaggagggcgccgacgccctgggccctgatggcaaattgctccactcggatgctttcccgccaccccgcgtggtggatacactgggagctggagacaccttcaatgcctccgtcatcttcagcctctcccaggggaggagcgtgcaggaagcactgagattcgggtgccaggtggccggcaagaagtgtggcctgcagggctttgatggcatcgtgtgagagcaggtgccggctcctcacacaccatggagactaccattgcggctgcatcgccttctcccctccatccagcctggcgtccaggttgccctgttcaggggacagatgcaagctgtggggaggactctgcctgtgtcctgtgttccccacagggagaggctctggggggatggctgggggatgcagagcctcagagcaaataaatcttcctcagagccagcttctcctctcaatgtctgaactgctctggctgggcattcctgaggctctgactcttcgatcctccctctttgtgtccattccccaaattaacctctccgcccaggcccagaggaggggctgcctgggctagagcagcgagaagtgccctgggcttgccaccagctctgccctggctggggaggacactcggtgccccacacccagtgaacctgccaaagaaaccgtgagagctcttcggggccctgcgttgtgcagactctattcccacagctcagaagctgggagtccacaccgctgagctgaactgacaggccagtggggggcaggggtgcgcctcctctgccctgcccaccagcctgtgatttgatggggtcttcattgtccagaaatacctcctcccgctgactgccccagagcctgaaagtctcacccttggagcccaccttggaattaagggcgtgcctcagccacaaatgtgacccaggatacagagtgttgctgtcctcagggaggtccgatctggaacacatattggaattggggccaactccaatatagggtgggtaaggccttataatgtaaagagcatataatgtaaagggctttagagtgagacagacctggattcaaatctgccatttaattagctgcatatcaccttagggtacagcacttaacgcaatctgcctcaatttcttcatctgtcaaatggaaccaattctgcttggctacagaattattgtgaggataaaatcatatataaaatgcccagcatgatgaaaaaaaaaaaaaaaaaa

SEQ ID NO: 4 - protein, 298 aa, Homo sapiens
MEEKQILCVGLVVLDVISLVDKYPKEDSEIRCLSQRWQRGGNASNSCTVLSLLGAPCAFMGSMAPGHVADFLVADFRRRGVDVSQVAWQSKGDTPSSCCIINNSNGNRTIVLHDTSLPDVSATDFEKVDLTQFKWIHIEGRNASEQVKMLQRIDAHNTRQPPEQKIRVSVEVEKPREELFQLFGYGDVVFVSKDVAKHLGFQSAEEALRGLYGRVRKGAVLVCAWAEEGADALGPDGKLLHSDAFPPPRVVDTLGAGDTFNASVIFSLSQGRSVQEALRFGCQVAGKKCGLQGFDGIV